(19) United States
(12) Patent Application Publication
(10) Pub. No.: US 2025/0338856 A1
(43) Pub. Date: Nov. 6, 2025

(54) BACTERIAL COMPOSITION AND A METHOD OF USING SAME

(71) Applicant: MICROBIOME LTD., Tel Mond (IL)

(72) Inventors: Yossi COHEN, Herzliya (IL); Yulimar Castro MOLINA, Merida (VE)

(21) Appl. No.: 18/870,295

(22) PCT Filed: May 30, 2023
(86) PCT No.: PCT/IL2023/050554

§ 371 Date: Nov. 27, 2024

Related U.S. Application Data
(60) Provisional application No. 63/346,974, filed on May 30, 2022.

(51) Int. Cl. A01N 63/22; A01N 63/20; A01P 3/00; C12N 1/20; C12R 1/01; C12R 1/065; C12R 1/125; C12R 1/41

(52) U.S. Cl. CPC A01N 63/22; A01N 63/20; A01P 3/00; C12N 1/205; C12R 2001/01; C12R 2001/065; C12R 2001/125; C12R 2001/41

(57) ABSTRACT

There is provided a synthetic composition including: (a) bacterial consortium including: (i) at least three bacterial species belonging to the genus Bacillus; and (ii) at least three bacterial species belonging to the genus Lacticaseibacillus; and (b) an acceptable carrier, as well as a method of using same, such as for preventing or treating a fungal infection in a plant or a plant part.

### REFERENCE TO AN ELECTRONIC SEQUENCE LISTING

The contents of the electronic sequence listing (MCRB-P-001-PCT ST26.xml; size: 6,274 bytes; and date of creation: May 28, 2023) is herein incorporated by reference in its entirety.

### CROSS-REFERENCE TO RELATED APPLICATIONS

This application claims the benefit of priority of U.S. Provisional Application No. 63/346,974, titled “BACTERIAL COMPOSITION AND A METHOD OF USING SAME”, filed 30 May 2022, the contents of which are incorporated herein by reference in their entirety.

### FIELD OF INVENTION

The present invention relates to inter alia synthetic bacterial composition, and a method of using the same, such as for controlling fungal infections in plant or plan part.

### BACKGROUND

Plants interact with a broad range of microbial organisms throughout their lifetime, including pathogens. In order to support their health status and cope with pathogen challenges, plants produce a large array of chemical compounds. Phytopathogenic fungi, such as Botrytis spp. including B. cinerea are necrotrophic pathogens, causing rot on above-ground organs, with a wide host range of more than 1,400 plant species.

Phytopathogens, such as Botrytis cinerea, causes huge losses in crops during growth and storage of fruits, vegetables and cut flowers. The broad host range of B. cinerea is due to the wide range of virulence factors, including lytic enzymes and toxins, as well as factors which reduce host defense and alter levels of reactive oxygen species (ROS).

There is still a great need for anti-fungal compositions that are both effective in controlling infections, as well as safe for human consumption, and the environment.

### SUMMARY

The present invention, in some embodiments, is based, in part, on the findings that synthetic compositions comprising: (a) a bacterial consortium comprising: (i) at least three bacterial species belonging to the genus Bacillus; and (ii) at least three bacterial species belonging to the genus Lacticaseibacillus; and (b) an acceptable carrier, were found to be highly effective against fungi, including Penicillium digitatum (e.g., “green mold”).

According to a first aspect, there is provided a synthetic composition comprising: (a) bacterial consortium comprising: (i) at least three bacterial species belonging to the genus Bacillus; and (ii) at least three bacterial species belonging to the genus Lacticaseibacillus; and (b) an acceptable carrier.

According to another aspect, there is provided a method for preventing or treating a fungal infection in a plant or a plant part, the method comprising contacting the plant or plant part with an effective amount of the synthetic composition disclosed herein, thereby preventing or treating a fungal infection in the plant or plant part.

In some embodiments, the at least three bacterial species belonging to the Bacillus, comprise: Bacillus subtilis, Bacillus amyloliquefaciens, and Bacillus licheniformis.

In some embodiments, the at least three bacterial species belonging to the genus Lacticaseibacillus, comprise: Lacticaseibacillus casei, Lacticaseibacillus paracasei, and Lacticaseibacillus rhamnosus.

In some embodiments, the synthetic composition further comprises at least one first additional bacterial species belonging to any one of: (a) the genus Latilactobacillus; (b) the genus Lactiplantibacillus; (c) the genus Lactococcus; (d) the genus Limosilactobacillus; or (e) any combination of (a) to (d).

In some embodiments, the: (a) at least one first additional bacterial species belonging to the genus Latilactobacillus, comprises Latilactobacillus sakei; (b) at least one first additional bacterial species belonging to the genus Lactiplantibacillus, comprises Lactiplantibacillus plantarum; (c) at least one first additional bacterial species belonging to the genus Lactococcus, comprises Lactococcus lactis; and (d) at least one first additional bacterial species belonging to the genus Limosilactobacillus, comprises Limosilactobacillus fermentum.

In some embodiments, the synthetic composition further comprises at least one second additional bacterial species belonging to any one of: (a) the genus Rhizobium; (b) the genus Azospirillum; (c) the genus Azobacter; and (d) any combination of (a) to (c).

In some embodiments, the: (a) at least one second additional bacterial species belonging to the genus Rhizobium, comprises Rhizobium tropici AM001, Rhizobium etli AM015, or both; (b) at least one second additional bacterial species belonging to the genus Azospirillum, comprises Azospirillum sp AS101; and (c) at least one second additional bacterial species belonging to the genus Azobacter, comprises Azobacter vinelandii AS122.

In some embodiments, the synthetic composition comprises the at least three bacterial species belonging to the genus Bacillus and the at least three bacterial species belonging to the genus Lacticaseibacillus at colony forming unit (CFU) to CFU ratio ranging from 10,000:1 to 1,000:1.

In some embodiments, the synthetic composition comprises the Bacillus subtilis, Bacillus amyloliquefaciens, and Bacillus licheniformis, at a CFU ranging from 1×10⁹ to 1×10¹², 1×10⁶ to 1×10¹⁰, and 1×10⁵ to 1×10⁸, respectively.

In some embodiments, the synthetic composition comprises Lacticaseibacillus casei, Lacticaseibacillus paracasei, and Lacticaseibacillus rhamnosus, at a CFU ranging from 1×10³ to 1×10⁶, 1×10³ to 1×10⁶, and 1×10⁵ to 1×10⁸, respectively.

In some embodiments, the synthetic composition comprises any one of: (a) Latilactobacillus sakei at a CFU ranging from 1×10⁵ to 1×10⁸; (b) Lactiplantibacillus plantarum at a CFU ranging from 1×10⁶ to 1×10⁹; (c) Lactococcus lactis at a CFU ranging from 1×10⁶ to 1×10⁹; and (d) Limosilactobacillus fermentum at a CFU ranging from 1×10⁴ to 1×10⁷.

In some embodiments, the synthetic composition comprises any one of: (a) Rhizobium tropici AM001, Rhizobium etli AM015, or both at a CFU ranging from 1×10⁷ to 1×10¹⁰; (b) Azospirillum sp AS101 at a CFU ranging from 1×10⁵ to 1×10⁸; and (c) Azobacter vinelandii AS122 at a CFU ranging from 1×10⁷ to 1×10¹¹.

In some embodiments, the synthetic composition comprises Rhizobium tropici AM001 and Rhizobium etli AM015 at a CFU: CFU ratio of 1:1.

In some embodiments, the carrier comprises: Citrus Aurantium Amara extract, Citrus reticulata extract, Citrus aurantium sinensis extract, Ethylhexyl glycerin, Phenoxyethanol, or any combination thereof.

In some embodiments, the carrier comprises: Citrus Aurantium Amara extract, Citrus reticulata extract, Citrus aurantium sinensis extract, Ethylhexyl glycerin, and Phenoxyethanol.

In some embodiments, the carrier further comprises any one of: Brassica oleraceae var. capitata extract, Microbial alginate, and both.

In some embodiments, the synthetic composition further comprises a glycoside hydrolase.

In some embodiments, the glycoside hydrolase is myrosinase.

In some embodiments, the synthetic composition comprises the carrier in a weight per weight ratio (w/w) ranging from 8% to 15% of the composition.

In some embodiments, the synthetic composition is a fungicidal composition.

In some embodiments, the synthetic composition is formulated for dipping, soaking, spraying, coating, or any combination thereof, to a plant or a plant part.

In some embodiments, the fungal infection is induced by a fungus being selected form the group consisting of: Botrytis cinérea, Mycosphaerella fragarie, Colletotrichum sp., Alternaria alternata, Penicillium italicum, Phytophthora citroththora, Penicillium digitatum, Phomopsis sp., Diplodia sp., Rhizopus nigricans, Sphaceloma persea, Cercospora purpura, Cladosporium herbarum, Neofabraea alba, Diplodia seriata, Phacidiopycnis washingtonensis, Rhizopus stolonifer/Mucor spp., Monilinia fructicola, Venturia inaequalis, Stemphillium sp., and any combination thereof.

In some embodiments, the plant part is selected from the group consisting of: a fruit, a leaf, a flower, a seed, a tuber, a bulb, and any combination thereof.

In some embodiments, the contacting comprises preharvest contacting, post-harvest contacting, or both.

In some embodiments, the contacting is postharvest contacting.

In some embodiments, post harvesting contacting is in a storage facility.

In some embodiments, the contacting is by dipping, soaking, spraying, coating, washing, humidifying, fogging, or any combination thereof, the plant or plant part with the composition.

In some embodiments, the preventing or treating comprises inhibiting any one of: attachment, germination, penetration, host tissue colonization, growth, proliferation, metabolism, spore release, hyphae production, hyphae penetration, or any combination thereof, of a fungus inducing the fungal infection.

In some embodiments, the preventing or treating comprises increasing shelf life of the plant or plant part by at least 20%.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a photograph and a table showing morphotype of colonies isolated from rhizosphere soil.

FIG. 2 includes a photograph of an agarose gel showing evaluation of the integrity of bacterial DNA extracted from 5 colonies isolated from rhizospheric soil. The total genomic DNA band, an intense band, is observed in bacterial colonies 2, 3, and 5, indicating good DNA integrity.

FIG. 3 includes a photograph of an agarose gel showing amplification of the 16S gene of the isolated bacterial colonies. MM-molecular marker. An amplification product of 1,500 bp is observed. The best amplification of the 16S gene was observed in colonies 3, 4, and 5.

FIG. 4 includes a phylogenetic tree of the 16S rRNA gene sequences of the strains identified as Bacillus subtilis concerning sequences of reference strains from the GenBank. Generated with the Mega 6 program. Bootstrap values are based on 1,000 replicates.

FIG. 5 includes a photograph of an enzyme-linked immunosorbent assay (ELISA) microplate showing result of the production of indole-3-acetic acid (IAA) by the Bacillus subtilis Q135 strain.

FIG. 6 include a photograph of petri dishes showing the pathogenic fungus Fusarium oxysporum either cultured alone (left) or under in vitro confrontation with the bacterium Bacillus subtilis Q135 (right).

FIG. 7 include a photograph showing a pre-inoculum in of three bacteria of the genus Lacticaseibacillus in saline solution (0.85%).

FIG. 8 includes a photograph showing a microbial consortium comprising three strains of Lacticaseibacillus in a cell suspension.

FIG. 9 includes a graph showing kinetics of growth of a microbial consortium of three species of Lacticaseibacillus in three formulations of tobacco extract.

FIG. 10 includes a photograph showing a compatibility test of 11 species of bacteria from 6 different genera (performed in a triplicate).

FIG. 11 includes a photograph showing reaction of the urease enzyme in lactic acid bacteria. Bacteria of the Bacillus genus showed a positive response, as indicated by a color change from red to fuchsia.

FIG. 12 includes a photograph of an in vitro assay with Simmons citrate agar medium. Bacteria of the Bacillus genus were found to be favorable according to this test.

FIG. 13 includes photographs before (left) and after (right) a nitrate reduction test for lactic acid bacteria and bacteria of the Bacillus genus.

FIG. 14 includes a graph showing growth of the bacterial consortium.

FIG. 15 includes a photograph showing root nodules of an Alfalfa plant (Medicago sativa).

FIG. 16 includes a photograph showing a pure culture of Rhizobium sp, grown in Petri dishes with YMA medium, after five days of incubation at 30° C.

FIG. 17 includes a micrograph and a photograph showing the identification of morphological aspects of Rhizobium sp. Irregularly shaped colonies, with smooth border, cream color, and shiny appearance, were observed (left). Gross morphology before and after 7 days of incubation in acid reaction in YMA+Bromothymol blue medium are presented (right).

FIG. 18 includes a photograph of HCN production test in bacterial species of the formulation.

FIG. 19 includes a photograph of a phosphate solubilization test in bacterial strains that make up the microbial consortium. The strains: B. subtilis; A. brasilense, and R. etli were positive for this test (pointed by arrows).

FIG. 20 includes a graph showing growth of the microbial consortium in two concentrations of the formulation (1.5% and 2.7%).

FIG. 21 includes a photograph showing pure colonies of Azotobacter sp. grown in Petri dishes with NF medium after five days of incubation.

FIG. 22 includes a photograph of an agarose gel wherein PCR products were resolved, evidencing integrity of the DNA in different concentrations.

FIG. 23 includes a photograph of a micropleasr showing GA3-producing bacteria as a growth promotion mechanism. B. subtilis; B. amyloliquefaciens; B. licheniformis; A. brazilense; A. vinelandii; and L. rhamnosus showed positive reaction.

FIG. 24 includes a photograph of Petri dishes showing positive potassium solubilization reaction by bacteria of the microbial consortium. B subtilis Q135; B. amyloliquefaciens; B. licheniformis; A. brazilense KF2212; A. vinelandii AS122; L. rhamnosus; and Rhizobium etli AM001 showed positive reaction.

FIGS. 25A-25B include photographs of Petri dishes showing production of siderophores by bacterial species of the microbial consortium, used as a mechanism of action for the control of phytopathogens (25A). Production of Azotobacter vinelandii siderophos after 15 days of growth, evidencing their potential (25B).

FIG. 26 includes a photograph and a micrograph showing a device (upper panel) and a viability count (lower panel) of the bacterial inoculum of the consortium used in the encapsulation.

FIG. 27 includes a photograph of a petri dish of a compatibility test of 4 Bacillus species, grown in LB medium.

FIG. 28 include a photograph of petri dish of an In vitro assay of antagonistic activity of 4 Bacillus species on anthracnose (Colletotrichum gloeosporioides).

FIG. 29 include a photograph of a gel visualizing the amplification of the 16S gene of the isolated strains.

FIG. 30 includes photographs of petri dished showing inhibition of Botrytis cinerea by the antagonistic activity of the microbial consortium, after 8 days of incubation

FIG. 31 includes photographs of petri dishes showing inhibition of Colletotrichum gloeosporioides by the antagonistic activity of the microbial consortium, after 8 days of incubationz.

FIG. 32 includes a vertical bar graphs showing the logarithm of the number of colony forming units (CFU) of the microbial consortium in the formulation at 72, 96 and 120 hours.

### DETAILED DESCRIPTION

### Composition

According to some embodiments, there is provided a composition comprising a bacterial consortium. In some embodiments, the composition is a synthetic or an artificial composition.

As used herein, the terms “synthetic” or “artificial” are interchangeable and refer to a manmade composition, such as in vitro grown, cultured, or formulated composition, e.g., in a lab or a comparable facility.

In some embodiments, the composition comprises a bacterial consortium comprising at least three bacterial species belonging to the genus Bacillus.

In some embodiments, the composition comprises a bacterial consortium comprising at least three bacterial species belonging to the genus Lacticaseibacillus.

In some embodiments, the composition comprises a bacterial consortium comprising: (i) at least three bacterial species belonging to the genus Bacillus; and (ii) at least three bacterial species belonging to the genus Lacticaseibacillus.

In some embodiments, the composition comprises: (a) a bacterial consortium comprising: (i) at least three bacterial species belonging to the genus Bacillus; and (ii) at least three bacterial species belonging to the genus Lacticaseibacillus; and (b) an acceptable carrier.

In some embodiments, the at least three bacterial species belonging to the Bacillus, comprises Bacillus subtilis.

In some embodiments, the at least three bacterial species belonging to the Bacillus, comprises Bacillus amyloliquefaciens.

In some embodiments, the at least three bacterial species belonging to the Bacillus, comprises Bacillus licheniformis.

In some embodiments, the at least three bacterial species belonging to the Bacillus, comprises Bacillus subtilis and Bacillus amyloliquefaciens.

In some embodiments, the at least three bacterial species belonging to the Bacillus, comprises Bacillus subtilis and Bacillus licheniformis.

In some embodiments, the at least three bacterial species belonging to the Bacillus, comprises Bacillus amyloliquefaciens and Bacillus licheniformis.

In some embodiments, the at least three bacterial species belonging to the Bacillus, comprises Bacillus subtilis, Bacillus amyloliquefaciens, and Bacillus licheniformis.

In some embodiments, the at least three bacterial species belonging to the genus Lacticaseibacillus, comprises Lacticaseibacillus casei.

In some embodiments, the at least three bacterial species belonging to the genus Lacticaseibacillus, comprises Lacticaseibacillus paracasei.

In some embodiments, the at least three bacterial species belonging to the genus Lacticaseibacillus, comprises Lacticaseibacillus rhamnosus.

In some embodiments, the at least three bacterial species belonging to the genus Lacticaseibacillus, comprises Lacticaseibacillus casei and Lacticaseibacillus paracasei.

In some embodiments, the at least three bacterial species belonging to the genus Lacticaseibacillus, comprises Lacticaseibacillus casei and Lacticaseibacillus rhamnosus.

In some embodiments, the at least three bacterial species belonging to the genus Lacticaseibacillus, comprises Lacticaseibacillus paracasei and Lacticaseibacillus rhamnosus.

In some embodiments, the at least three bacterial species belonging to the genus Lacticaseibacillus, comprise Lacticaseibacillus casei, Lacticaseibacillus paracasei, and Lacticaseibacillus rhamnosus.

In some embodiments, the composition further comprises at least one additional bacterial species. In some embodiments, the composition further comprises at least a plurality of types of additional bacterial species. In some embodiments, the plurality of types of additional bacterial species comprises at least one first additional bacterial species and at least one second bacterial species.

In some embodiments, the at least one first additional bacterial species belongs to the genus Latilactobacillus.

In some embodiments, the at least one first additional bacterial species belongs to the genus Lactiplantibacillus.

In some embodiments, the at least one first additional bacterial species belongs to the genus Lactococcus.

In some embodiments, the at least one first additional bacterial species belongs to the genus Limosilactobacillus.

In some embodiments, the at least one first additional bacterial species belongs to any one of: (a) the genus Latilactobacillus; (b) the genus Lactiplantibacillus; (c) the genus Lactococcus; the genus Limosilactobacillus; or (e) any combination of (a) to (d).

In some embodiments, the at least one first additional bacterial species belonging to the genus Latilactobacillus, comprises Latilactobacillus sakei. In some embodiments, Latilactobacillus comprises Latilactobacillus sakei.

In some embodiments, the at least one first additional bacterial species belonging to the genus Lactiplantibacillus, comprises Lactiplantibacillus plantarum. In some embodiments, Lactiplantibacillus, comprises Lactiplantibacillus plantarum.

In some embodiments, the at least one first additional bacterial species belonging to the genus Lactococcus, comprises Lactococcus lactis. In some embodiments, Lactococcus, comprises Lactococcus lactis.

In some embodiments, the at least one first additional bacterial species belonging to the genus Limosilactobacillus, comprises Limosilactobacillus fermentum. In some embodiments, Limosilactobacillus, comprises Limosilactobacillus fermentum.

In some embodiments, the at least one second additional bacterial species belongs to the genus Rhizobium.

In some embodiments, the at least one second additional bacterial species belongs to the genus Azospirillum.

In some embodiments, the at least one second additional bacterial species belongs to the genus Azobacter.

In some embodiments, the at least one second additional bacterial species belongs to: (a) the genus Rhizobium (b) the genus Azospirillum; (c) the genus Azobacter; and any combination of (a) to (d).

In some embodiments, the at least one second additional bacterial species belonging to the genus Rhizobium, comprises Rhizobium tropici AM001, Rhizobium etli AM015, or both. In some embodiments, Rhizobium, comprises Rhizobium tropici AM001, Rhizobium etli AM015, or both.

In some embodiments, Rhizobium tropici AM001, Rhizobium etli AM015 are isolated from the Andean Cordillera of Mérida, Venezuela, and are characterized by: their ability to grow in a wide pH and temperature range, to produce and release various substances resulting from their metabolism, including lipochitooligosaccharides (LCO), and two flagellar systems.

In some embodiments, the at least one second additional bacterial species belonging to the genus Azospirillum, comprises Azospirillum sp AS101. In some embodiments, Azospirillum, comprises Azospirillum sp AS101.

In some embodiments, Azospirillum sp AS101 is isolated from the mountains of the Southern Towns of Mérida-Venezuela and is characterized by being capable of quickly adapting to a broad range of temperature and humidity conditions.

In some embodiments, the at least one second additional bacterial species belonging to the genus Azobacter, comprises Azobacter vinelandii AS122. In some embodiments, Azobacter, comprises Azobacter vinelandii AS122.

In some embodiments, Azobacter vinelandii AS122 isolated from mountain soils in the towns of the South of Mérida-Venezuela, and is characterized by: being physiologically stable bacterium, which adapts to adverse conditions of temperature, humidity, and pH; forming cysts which allows its stability and permanence for long periods, very efficient in colonization and antibiosis production, and/or constituting a source for obtaining alginate with better properties, e.g., compared with brown algae.

In some embodiments, the composition comprises at least three bacterial species belonging to the genus Bacillus and at least three bacterial species belonging to the genus Lacticaseibacillus at colony forming unit (CFU) to CFU ratio ranging from 10,000:1 to 1,000:1, 10,000:1 to 100:1, 10,000:1 to 10:1, 10,000:1 to 1:1, 100,000:1 to 100:1, 1,000,000:1 to 1,000:1, 1:1 to 1:100, or 1:1 to 1:1,000. Each possibility represents a separate embodiment of the invention.

In some embodiments, the composition comprises Bacillus at a CFU ranging from 1×10⁸ to 1×10¹², 1×10⁹ to 1×10¹³, 1×10⁹ to 1×10¹², 1×10¹⁰ to 1×10¹², 1×10¹¹ to 1×10¹², 1×10⁹ to 1×10¹¹, or 1×10⁸ to 1×10¹³. Each possibility represents a separate embodiment of the invention.

In some embodiments, the composition comprises Bacillus amyloliquefaciens 1×10⁶ to 1×10¹⁰, 1×10⁶ to 1×10⁷, 1×10⁶ to 1×10⁸, 1×10⁶ to 1×10⁹, 1×10⁶ to 1×10¹¹, or 1×10⁵ to 1×10⁹. Each possibility represents a separate embodiment of the invention.

In some embodiments, the composition comprises Bacillus licheniformis, at a CFU ranging from 1×10⁵ to 1×10⁸, 1×10⁵ to 1×10⁶, 1×10⁵ to 1×10⁷, 1×10⁵ to 1×10⁹, 1×10⁴ to 1×10⁸, or 1×10⁴ to 1×10⁹. Each possibility represents a separate embodiment of the invention.

In some embodiments, the composition comprises Lacticaseibacillus casei at a CFU ranging from 1×10³ to 1×10⁶, 1×10³ to 1×10⁴, 1×10³ to 1×10⁵, 1×10⁴ to 1×10⁶, 1×10³ to 1×10⁷, or 1×10⁴ to 1×10⁸. Each possibility represents a separate embodiment of the invention.

In some embodiments, the composition comprises Lacticaseibacillus paracasei at a CFU ranging from 1×10³ to 1×10⁶, 1×10³ to 1×10⁴, 1×10³ to 1×10⁵, 1×10⁴ to 1×10⁶, 1×10³ to 1×10⁷, or 1×10⁴ to 1×10⁸. Each possibility represents a separate embodiment of the invention.

In some embodiments, the composition comprises Lacticaseibacillus rhamnosus at a CFU ranging from 1×10⁵ to 1×10⁸, 1×10⁵ to 1×10⁶, 1×10⁵ to 1×10⁷, 1×10⁶ to 1×10⁸, 1×10⁶ to 1×10⁷, or 1×10⁴ to 1×10⁹. Each possibility represents a separate embodiment of the invention.

In some embodiments, the composition comprises Latilactobacillus sakei at a CFU ranging from 1×10⁵ to 1×10⁸, 1×10⁵ to 1×10⁶, 1×10⁵ to 1×10⁷, 1×10⁶ to 1×10⁸, 1×10⁶ to 1×10⁷, or 1×10⁴ to 1×10⁹. Each possibility represents a separate embodiment of the invention.

In some embodiments, the composition comprises Lactiplantibacillus plantarum at a CFU ranging from 1×10⁶ to 1×10⁹, 1×10⁶ to 1×10¹⁰, 1×10⁶ to 1×10⁷, 1×10⁶ to 1×10⁸, 1×10⁶ to 1×10¹¹, or 1×10⁵ to 1×10⁹. Each possibility represents a separate embodiment of the invention.

In some embodiments, the composition comprises Lactococcus lactis at a CFU ranging from 1×10⁶ to 1×10⁹, 1×10⁶ to 1×10¹⁰, 1×10⁶ to 1×10⁷, 1×10⁶ to 1×10⁸, 1×10⁶ to 1×10¹¹, or 1×10⁵ to 1×10⁹. Each possibility represents a separate embodiment of the invention.

In some embodiments, the composition comprises Limosilactobacillus fermentum at a CFU ranging from 1×10⁴ to 1×10⁷, 1×10⁴ to 1×10⁵, 1×10⁴ to 1×10⁶, 1×10⁵ to 1×10⁶, 1×10⁵ to 1×10⁷, 1×10⁶ to 1×10⁷, 1×10³ to 1×10⁷, or 1×10⁴ to 1×10⁸. Each possibility represents a separate embodiment of the invention.

In some embodiments, the composition comprises Rhizobium tropici AM001, Rhizobium etli AM015, or both, at a CFU ranging from 1×10⁷ to 1×10¹⁰, 1×10⁷ to 1×10⁸, 1×10⁷ to 1×10⁹, 1×10⁸ to 1×10¹¹, 1×10⁸ to 1×10⁹, 1×10⁸ to 1×10¹⁰, 1×10⁹ to 1×10¹⁰, or 1×10⁹ to 1×10¹¹. Each possibility represents a separate embodiment of the invention.

In some embodiments, the composition comprises Azospirillum sp AS101 at a CFU ranging from 1×10⁵ to 1×10⁸, 1×10⁵ to 1×10⁶, 1×10⁵ to 1×10⁷, 1×10⁶ to 1×10⁸, 1×10⁶ to 1×10⁷, or 1×10⁴ to 1×10⁹. Each possibility represents a separate embodiment of the invention.

In some embodiments, the composition comprises Azobacter vinelandii AS122 at a CFU ranging from 1×10⁷ to 1×10¹¹, 1×10⁷ to 1×10¹⁰, 1×10⁷ to 1×10⁸, 1×10⁷ to 1×10⁹, 1×10⁸ to 1×10¹¹, 1×10⁸ to 1×10⁹, 1×10⁸ to 1×10¹⁰, 1×10⁹ to 1×10¹⁰, or 1×10⁹ to 1×10¹². Each possibility represents a separate embodiment of the invention.

In some embodiments, the composition comprises Rhizobium tropici AM001 and Rhizobium etli AM015 at a CFU: CFU ratio ranging from 10:1 to 1:10, 8:1 to 1:8, 5:1 to 1:5, 4:1 to 1:4, 3:1 to 1:3, or 2:1 to 1:2. Each possibility represents a separate embodiment of the invention.

In some embodiments, the composition comprises Rhizobium tropici AM001 and Rhizobium etli AM015 at a CFU: CFU ratio of 1:1.

In some embodiments, the carrier is a natural carrier. In some embodiments, the carrier comprises natural compounds or agents. In some embodiments, the carrier consists of natural compounds or agents. In some embodiments, the carrier comprises or consists of extracts obtained or derived from natural compounds or agents, e.g., fruit or fruit material. In some embodiments, the carrier is an edible carrier. In some embodiments, the carrier is safe for a subject consumption, e.g., via digestion. In some embodiments, the carrier is safe for a human consumption, such as, but not limited to food. In some embodiments, the carrier is an agriculturally acceptable carrier.

In some embodiments, the carrier comprises: Citrus Aurantium Amara extract, Citrus reticulata extract, Citrus aurantium sinensis extract, Ethylhexyl glycerin, Phenoxyethanol, or any combination thereof.

In some embodiments, the carrier comprises: Citrus Aurantium Amara extract, Citrus reticulata extract, Citrus aurantium sinensis extract, Ethylhexyl glycerin, and Phenoxyethanol.

In some embodiments, the carrier further comprises: Brassica oleraceae var. capitata extract, Microbial alginate, or both.

In some embodiments, the composition of the invention further comprises a glycoside hydrolase.

In some embodiments, the carrier further comprises a glycoside hydrolase.

In some embodiments, a glycoside hydrolase comprises myrosinase.

In some embodiments, the composition comprises a carrier in a weight per weight ratio (w/w) ranging from 8% to 9%, 8% to 10%, 8% to 11%, 8% to 12%, 8% to 13%, 8% to 14%, 8% to 15%, 9% to 15%, 10% to 15%, 12% to 15%, or 6% to 18%, of the composition. Each possibility represents a separate embodiment of the invention.

In some embodiments, the composition a fungicidal composition. In some embodiments, the composition is characterized by having or comprises a fungicidal activity.

In some embodiments, fungicidal activity comprises inhibition of fungal attachment, germination, penetration, host tissue colonization, or any combination thereof, killing fungus, inhibiting fungus replication, growth, DNA synthesis, spore formation and/or release, spore germination, spore growth, or any combination thereof.

In some embodiments, fungicidal activity comprises fungistatic activity.

In some embodiments, fungicidal activity comprises inhibition or reduction of fungal spore germination.

In some embodiments, the composition further comprises a compound having fungicidal activity, fungistatic activity, or both.

In some embodiments, the composition is an agricultural composition.

In some embodiments, the composition is an antibiotic composition.

In some embodiments, the composition is formulated for dipping, soaking, spraying, coating, or any combination thereof, to a plant or a plant part.

As used herein, the phrase “agriculturally acceptable” excipient or carrier is suitable for use in agriculture without undue adverse side effects to the plants, the environment, or to humans or animals who consume the resulting agricultural products derived therefrom commensurate with a reasonable benefit/risk ratio.

As used herein, the term “carrier”, “excipient”, or “adjuvant” refers to any component of a composition, that is not the active agent, such as, but not limited to the bacterial consortium disclosed herein.

### Methods of Use

According to some embodiments, there is provided a method for preventing or treating a fungal infection in a plant or a plant part.

In some embodiments, the method comprises contacting the plant or plant part with an effective amount of a synthetic composition, as disclosed herein, thereby preventing or treating a fungal infection in the plant or plant part.

In some embodiments, the method comprises contacting the plant or plant part with an effective amount of a synthetic composition comprising: (a) a bacterial consortium comprising: (i) at least three bacterial species belonging to the genus Bacillus; and (ii) at least three bacterial species belonging to the genus Lacticaseibacillus; and (b) an acceptable carrier.

In some embodiments, a fungal infection is induced by a fungus being selected form: Botrytis cinérea, Mycosphaerella fragarie, Colletotrichum sp., Alternaria alternata, Penicillium italicum, Phytophthora citroththora, Penicillium digitatum, Phomopsis sp., Diplodia sp., Rhizopus nigricans, Sphaceloma persea, Cercospora purpura, Cladosporium herbarum, Neofabraea alba, Diplodia seriata, Phacidiopycnis washingtonensis, Rhizopus stolonifer/Mucor spp., Monilinia fructicola, Venturia inaequalis, Stemphillium sp., or any combination thereof.

In some embodiments, a plant being treated according to the herein disclosed method is selected from the table 1, hereinbelow.

TABLE 1
Plants suitable for prevention or treatment
according to the method of the invention.

| | Family | Genus | Species |
|---|---|---|---|
| Orange | Rutaceae | Citrus | sinensis, aurantium |
| Grape | Vitaceae | Vitis | vinifera |
| Apple | Rosaceae | Malus | domestica |
| Lemon | Rutaceae | Citrus | limon |
| Avocado | Lauraceae | Persea | americana |
| Strawberry/berries | Rosaceae | Fragaria | ananassa |
| Peach | Rosaceae | Prunus | persica |
| Apricot | Rosaceae | Prunus | armeniaca |
| Cambur/banana | Musaceae | Musa | paradisiaca |
| Kaki (Caqui) | Ebenaceae | Diospyrus | kaki |
| Mandarin | Rutaceae | Citrus | reticulata |
| Mango | Anacardiaceae | Mangifera | indica |
| Pear | Rosaceae | Pyrus | communis |
| Kiwi | Actinidiaceae | Actinidia | deliciosa |
| Cantaloupe | Cucurbitaceae | Cucumis | melo |
| papaya/milky | Caricaceae | Carica | papaya |
| Soursop | Annonaceae | Annona | muricata Linn |
| Guava | Myrtaceae | Psidium | guajaba |
| Pepper | Solanaceae | Capsicum | annuum |
| Sweet potato | Solanaceae | Solanum | tuberosum |
| Lettuce | Asteraceae | Lactuca | sativa |
| Carrot | Apiaceae | Daucus | carota |
| Onion | Amaryllidaceae | Allium | cepa |
| Tomato | Solanaceae | Solanum | lycopersicum |
| Cucumber | Cucurbitaceae | Cucumis | sativus |
| Cauliflower | Brassicaceae | Brassica | oleracea |
| Olive | Oleaceae | Olea | europaea |
| Cherry | | Prunus | e.g., avium, cerasus |

In some embodiments, plant part is selected from: a fruit, a leaf, a flower, a seed, a tuber, a bulb, or any combination thereof.

In some embodiments, contacting comprises preharvest contacting, post-harvest contacting, or both.

In some embodiments, contacting is postharvest contacting.

In some embodiments, post harvesting contacting is in a storage facility.

In some embodiments, contacting a plant or plant part with the herein disclosed composition is by dipping, soaking, spraying, coating, washing, humidifying, fogging, or any combination thereof.

In some embodiments, preventing or treating comprises inhibiting: attachment, germination, penetration, host tissue colonization, growth, proliferation, metabolism, spore release, hyphae production, hyphae penetration, or any combination thereof, of a fungus inducing a fungal infection.

In some embodiments, preventing or treating comprises increasing shelf life of a plant or plant part.

In some embodiments, increasing is by at least 5%, 10%, 20%, 50%, 100%, 250%, 400%, 500%, 700%, 1,000%, or any value and range therebetween. Each possibility represents a separate embodiment of the invention. In some embodiments, increasing is by 5-100%, 50-250%, 100-500%, 150-650%, 200-750%, or 200-1,000%. Each possibility represents a separate embodiment of the invention.

In some embodiments, inhibiting or reducing germination of spores comprises: reducing the number of normally elongated tubes of the spores, increasing the number of short germination tubes of the spores, reducing the frequency of germinating spores, increasing the number of damaged tubes of the spores, increasing the number of spores having no tubes, or any combination thereof.

In some embodiments, inhibiting or reducing comprises at least 5%, at least 15%, at least 25%, at least 50%, at least 65%, at least 75%, at least 85%, at least 95%, at least 97%, at least 99%, or 100% inhibiting or reducing, or any value and range therebetween. Each possibility represents a separate embodiment of the invention. In some embodiments, inhibiting or reducing comprises 5-50%, 10-90%, 20-99%, or 5-100% inhibiting or reducing. Each possibility represents a separate embodiment of the invention.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm+100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Example 1

The methodology used for isolating bacteria of the genus Bacillus in the soil is presented, with characteristics that promote plant growth, that act as biological control, and their ability to grow in sugarcane molasses to produce acid: indole-3-acetic acid (IAA), simulating molasses as a vehicle for a potential bacteria-based product.

### Laboratory Bioassays

### Isolation of Bacteria of the Genus Bacillus sp.

Six rhizospheric soil samples were collected at equidistant points from 6 coffee plants. Then, the samples were mixed to obtain a composite sample stored in polypropylene bags and kept at 4° C. until processing. Subsequently, 5 grams of soil were submerged in flasks with 200 ml of a sterile aqueous solution of 0.05% tween 20, making five flasks orbitally shaken at 160 rpm at room temperature for 1 h. For the isolation of bacteria, serial dilutions were prepared up to 10-4, where 0.1 mL of the 10-2, 10-3, and 10-4 dilutions were cultured by extension in plates with Luria Bertani (LB) culture medium (Table 1. Composition of the LB medium) and were incubated at 30° C. for 48 hours, following the methodology of Prescott et al. (2002).

TABLE 1
Composition of the Luria Bertani (LB) medium

| Components | Concentration g · l⁻¹ |
|---|---|
| Tryptone | 10.00 |
| Yeast extract | 10.00 |
| NaCl | 5.00 |
| pH | 7.0 |
| Agar | 15.00 |

The medium was sterilized in an autoclave at 121° C. for 20 minutes.

### Calculation of Colony-Forming Units (CFU)

After 48 hours in incubation, the colony-forming units were calculated for 5 grams of soil using equation 1.

$$CFU \cdot {ml}^{- 1} \cdot {g}_{( dry soil )}^{- 1} = \frac{( Number of colonies ) \cdot ( Dilution Factor )}{( ml of sowing ) \cdot ( grams of dry soil )} . \qquad Equation 1$$

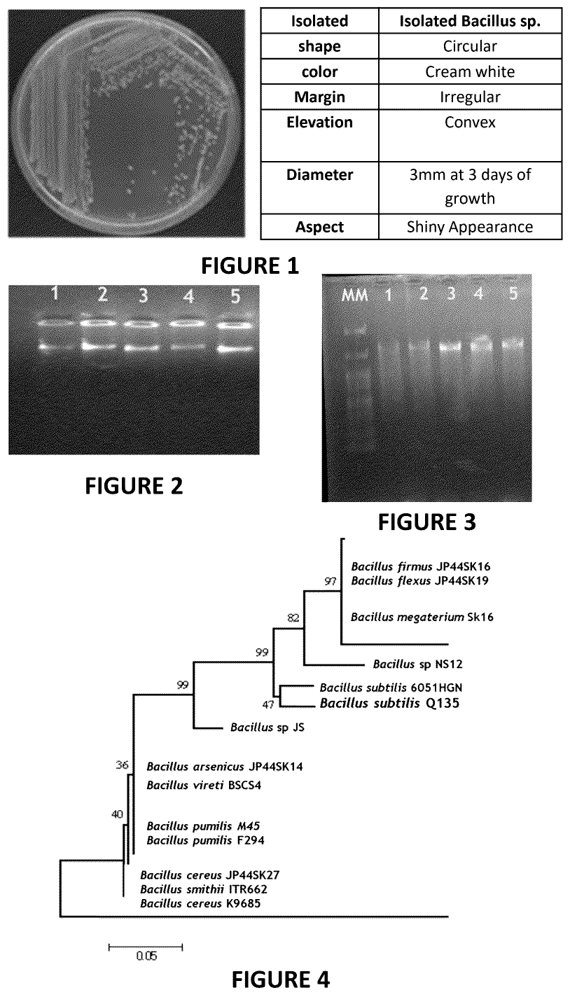

Subsequently, colonies with morphotypes similar to Bacillus sp. were isolated with a platinum loop, considering the colony's diameter, shape, texture, color, and edge (FIG. 1), following the methodology of Smibert and Krieg (1981), and seeded by exhaustion in the same LB medium. Likewise, the isolated strain was cultivated several times until purifying and stored at 4° C.,

### Molecular Identification of the Isolated Bacterial Strain

One of the purified bacterial colonies was seeded in test tubes containing 5 mL of liquid medium (LB) for 24 hours under constant agitation to then carry out the DNA extraction process using the SDS (Sodium Dodecyl Sultafo) method, following the methodology of Sambrook and Russell (2001). The integrity of the extracted DNA was determined by 1% agarose gel electrophoresis (FIG. 2). The DNA concentrations obtained were calculated by absorbance at 260 nm and 280 nm wavelengths using a Nanodrop 2000C.

### Amplification of the 16S Ribosomal Region

The taxonomic identification of the isolated bacterial strain was performed by amplifying the small subunit (16S) of the rDNA with the primers pA (5′-AGAGTTTGATCCTGGCTCAG-3′; SEQ ID NO: 1) and pc5B (5′-TACCTTGTTACGACTT-3′; SEQ ID NO: 2) according to Kuske et al. (1997). PCR reactions were performed in a total volume of 25 AL containing 0.5 μM of the primers, 1 U of recombinant Taq DNA polymerase (Fermentas, Lithuania), 0.2 mM of dNTPs, 1× enzyme buffer, 1.8 mM of MgCl₂, 1 μL of DNA (50 ng μL⁻¹) and sterile ultrapure water. The amplification was performed in a T3 thermocycler (Biometra) with a program that consisted of an initial denaturation at 94° C. for 3 min, followed by 40 cycles of 94° C. for 30 s, 60° C. for 30 s, 72° C. for 1 min; and a final extension at 72° C. for 7 min. After amplification, 5 μL of the reaction products were taken to separate them by electrophoresis in 1.5% agarose gel (FIG. 3).

Analysis of the 16S rRNA Sequences

The phylogenetic analysis of the nucleotide sequences of the bacteria was carried out using bioinformatics programs. The sequences were edited with the Bioedit program (Hall, 2005) and compared with the sequences deposited in the GenBank of the NCBI (National Center for Biotechnology Information) using the BlastN program (Altschul et al., 1997). The sequences were aligned with the Clustal W program (Thompson et al., 1994). Finally, a phylogenetic tree was created for the sequences of the 16S gene strains identified as Bacillus concerning sequences of GenBank reference strains (FIG. 4). For this, we used the Neighbor-Joining test (Saitou & Nei, 1987) and the phylogeny test with the Kimura2-parameters replacement model, 1,000 replicates, and Bootstrap method (Kimura, 1980) using the Mega 6 program (Tamura et al., 2013).

Production of Indole-3-Acetic Acid (IAA) by the Bacillus subtilis Strain

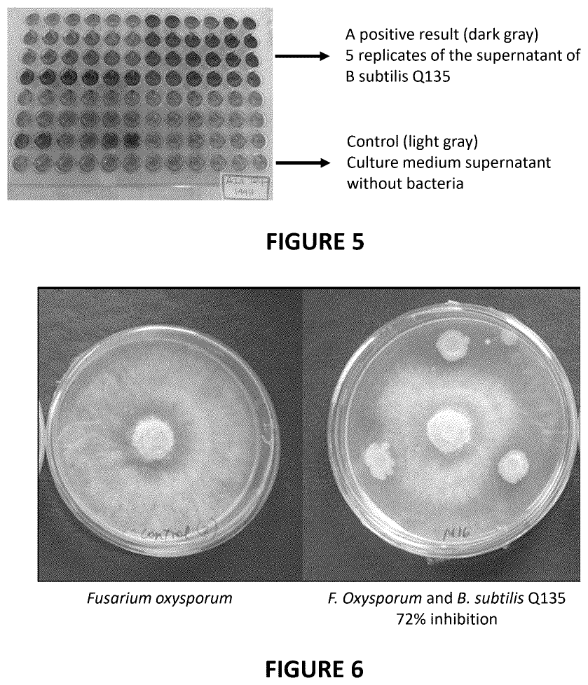

It was carried out using a qualitative and specific colorimetric method, which characterizes the production of phytohormone, described by Duca et al., 2014. A colony of the bacterial strain was inoculated in 5 ml of 10% Tryptic Soy Broth (TSB), supplemented with 5 mM L-tryptophan as a precursor for synthesizing indole-acetic acid, and incubated at 30° C., without light, for 24 hours. After the growth period, the culture was homogenized, transferred to sterilized centrifuge tubes, and spun at 10,000 rpm for 10 minutes. Aliquots of 500 AL of the supernatant were separated and transferred to Eppendorf-type tubes containing 500 μL of Salkowski's reagent (1 mL of 2% 0.5 M FeCl₃ in 49 mL of 35% perchloric acid) and incubated for 30 minutes. At 30° C., in the absence of light. Subsequently, a 300 μl aliquot was transferred to an Elisa plate (FIG. 5). The change from yellow to pink color is a positive result, and the intensity of the pink color is directly proportional to the concentration of indole-3-acetic acid produced by the bacteria and present in the medium.

Inhibition of In Vitro Growth of Fusarium oxysporum by Bacillus subtilis Q135

The inoculum of the pathogenic fungus Fusarium oxysporum included discs of mycelium grown in a Potato-Dextrose-Agar (PDA) medium with a diameter of 0.7 cm from a culture of 6 days of growth at a temperature of 28° C. The bacterium (B. subtilis Q135) was prepared from a colony with 48 hours of growth in nutrient broth incubated at 26° C. It was centrifuged at 4,000 rpm for 10 min, and the pellet was resuspended in sterile saline solution (0.89% w/w NaCl). Cell counting was performed in a Neubauer chamber, and the final inoculum was prepared at a cell density of 1×10⁻⁸ cells ml-1 in sterile saline solution.

### Confrontation or Dual Culture in PDA Medium

Two 10 μl drops of the bacterium Bacillus subtilis Q135 were seeded ˜2.5 cm from the Fusarium oxysporum disc is Petri dishes with a PDA culture medium. As a control, only sterile saline solution was seeded, according to the methodology of El-Sayed et al., (2014). The experiment was performed in a triplicate, and after 24 h at 30° C., a 0.7 cm diameter PDA disc containing the pathogen was placed in the center of each plate. The plates were incubated at 27° C. for eight days, recording daily observations. The diameters of the F. oxysporum colonies were measured with a ruler, and the inhibition percentage in the presence of Bacillus subtilis Q135 was calculated with equation 2, according to the methodology of Yu et al. (2011).

$$\% inhibition = ( 1 - \frac{Diameter of each treatment}{Diameter of the control} ) \times 100. \qquad Equation 2$$

Results of the In Vitro Growth Inhibition of Fusarium oxysporum by Bacillus subtilis Q135

The Bacillus subtilis Q135 strain showed the ability to inhibit the pathogenic fungus Fusarium oxysporum under in vitro conditions. In the test carried out with three repetitions, it was possible to determine that the strain B. subtilis Q135 inhibited the growth of F. oxysporum by approximately 72% (FIG. 6).

The antagonistic activity of Bacillus sp. is due to several mechanisms such as competition for colonization of the rhizosphere or fruit surfaces, production of lipopeptides such as Iturin, Fengycin, and Surfactin, and production of lytic enzymes according to Chang et al. (2010).

Use of Molasses as a Carbon Source for the Growth of B subtilis Q135 and Optimization of IAA Production

The current invention utilizes an agro-industrial byproduct, such as molasses, for the formulation of an agricultural product with biological control and plant growth promotion properties, seeking to increase and optimize bacterial indole-3-acetic acid AIA production by B. subtilis Q135.

Preparation of Solutions with Different Concentrations of Molasses for the Growth of B. Subtilis Q135

Before the laboratory tests with the molasses, this cane byproduct's composition was analyzed to determine which compounds and quantities were present (Table 2).

TABLE 2
Composition of sugar cane molasses

| Constituents | Content % |
|---|---|
| Dry matter | 78 |
| Protein | 3 |
| Sucrose | 60-63 |
| Reducing sugars | 3-5 |
| Dissolved substances (different sugars) | 4-8 |
| Water | 16 |
| Ashes | 9 |

The evaluation of microbial growth and IAA production of B. subtilis Q135 was carried out in different molasses solutions: 15, 20, and 30% w/v, supplemented with yeast extract (as a nitrogen source), monopotassium phosphate (KH₂PO₄) and Tryptophan (for the case of IAA production), adjusting each solution to a pH of 5.2±0.1 according to the modified protocol of Ortiz et al. (2008) (Table 3), a commercial culture medium YPD (yeast extract, peptone, dextrose) was used as a control.

The molasses solutions and the commercial medium were inoculated with 5 mL of inoculum of B. subtilis Q135 with an initial concentration of 1×10⁷ cfu/mL, adjusted by reading in a spectrophotometer at an optical density of 600 nm, and were incubated at 30° C.±1 with stirring at 100 rpm, for 48 hours. Subsequently, samples were taken every 4 hours, measuring absorbance at 600 nm of biomass in triplicate to assess cell growth. An aliquot of each solution was taken 24 hours later to determine IAA production following the methodology described above.

TABLE 3
Composition of molasses solutions, used as culture medium

| Solution | Composition g/L |
|---|---|
| M15% | Molasses 15; Yeast Ext 1; KH₂PO₄ 0.5 + 0.5 tript |
| M20% | Molasses 20; Yeast Ext 4; KH₂PO₄ 1 + 0.5 tript |
| M30% | Molasses 30; Yeast Ext 6; KH₂PO₄ 2.5 + 0.5 tript |
| YPD | Medium YPD Yeast Ext 2.5; peptone 3; dextrose 5 + 0.5 |
| | trip |

Tryptophan (tript) = IAA inducer

Growth Results of B subtilis Q135 in Molasses Media

With the data obtained from the growth curves, kinetic parameters of the B. subtilis Q135 bacterium were determined, such as the yield or biomass concentration from Yx substrate (g/g); specific growth velocity μx (h-1) and doubling time td (h) following the Malthus equation (1798).

$$Yx / td = μ x . \qquad Equation 3$$

TABLE 4
Kinetic parameters of Bacillus subtilis Q135 bacterial cells

| | | | | Control |
|---|---|---|---|---|
| | M15% | M20% | M30% | (YDP) |
| Yx | 0.22 | 0.76 | 0.97 | 3.12 |
| td | 8.51 | 6.27 | 5.62 | 3.41 |
| μx | 0.201 | 0.104 | 0.112 | 0.084 |
| AIA | 0.56 | 1.57 | 1.94 | 7.16 |

Yx (g/g) = Concentration of cell biomass from substrate
μx (h − 1) = Specific cell growth rate
td (h) = Cell doubling time

When analyzing the data obtained from biomass yields in the three molasses solutions and the control (Table 4), it is observed that none of the concentrations presented a higher yield compared to the commercial culture medium YDP (Yx=3.12 g/g), this value being an efficient yield of bacterial cell biomass and optimal for its massification in formulations. Regarding the doubling time, the cell suspension decreased drastically as the hours passed; the concentration of M15% had the highest value (8.51), which shows that with molasses, in addition to needing more growth time, the bacterium has little biomass production. In the same way, the growth rate was also affected since it is directly proportional to the cell doubling time. Therefore, the bacterium could not reach the metabolic activity necessary to biosynthesize compounds such as indole-3-acetic acid AIA by not having optimal growth. In the control medium, the bacterium B. subtilis Q135 produced an optimal concentration of IAA (7.16 μg/mL).

In this way, it was determined that there are likely better vehicles than molasses to use in the matrix with bacterial inoculums since its instability in the contribution of carbon sources impairs the growth and development of endospores of the inoculum.

The afore-mentioned instability can be attributed to 2 reasons: the first, when centrifuging the molasses to remove the solids present in it, the amount of substrate could have decreased; therefore, higher concentrations should be tried; the second, the sterilization of the molasses, can lead to a decrease in the concentration of the carbohydrates present and caramelization of the sugars. Finally, it was also possible to observe that the sugar source contributes to the increase in contaminants in the formulation.

### Conclusions

For a formulation for agricultural use, the process of isolation and selection of the active agent must have several characteristics that promote plant growth and act as a biological control of phytopathogens. In the current study, a bacterium identified as Bacillus subtilis Q135 was isolated from a field of organic coffee cultivation, indicating that it can be used in crops and harvest products. Laboratory experiments have shown that the bacterium can inhibit the growth of phytopathogenic fungi. This strain helps considerably reduce the use of fertilizers and fungicides, which generate resistance in plant pathogenic fungi and considerably affect the environment. Using molasses as a vehicle did not efficiently produce cell biomass, and was highly prone to contamination.

### Example 2

The present invention further dicloses a formula based on 4 Bacillus strains identified as B. subtilis Q135, B. amyloliquefaciens, B. pumilus, and B. licheniformis, cultivated in an aloe vera matrix for the production of lipopeptides. Aspects such as the composition and pH of the solutions prepared from aloe vera gels were evaluated, as well as the characterization of the antagonistic mechanisms of the four bacteria, depending on the production of the inhibition of the growth of the anthracnose fungus (Colletotrichum gloeosporioides) by in vitro confrontations.

### Laboratory Bioassays

In vitro compatibility tests between Bacillus bacteria of different species used as a microbial consortium

The compatibility between the four species of the genus Bacillus, namely Bacillus subtilis Q135 strain, B. amyloliquefaciens, B. licheniformis, and B. pumilus was verified by comparing Petri dishes with LB medium to visualize the colonies' growth without causing inhibition. For this, the plate was divided into four parts, and the strains were planted, one next to the other, with a bacteriological loop in a linear manner, thus, forming confrontations; later, it was incubated at 26° C. for 48 hours, following the León methodology. et al. (2019). As shown in FIG. 27, neither strain inhibited the growth of the other, confirming that they are compatible with growing in the consortium.

### Test of Antagonistic Activity of Bacteria Against Plant Pathogens

The in vitro biocontrol potential of the 4 Bacillus species on the phytopathogen Colletotrichum gloeosporioides (anthracnose that affects many post-harvest fruits) was evaluated using the methodology of Ezziyyani et al. (2004b), which included confrontations in Petri dishes with half PDA. In each plate, 15 μL of the 1×10⁷ cell inoculum was placed in triplicate· mL-1 of each Bacillus at a distance of 3 cm from each other. After 24 h of incubation at 26° C., 7 mm diameter agar discs of the phytopathogen in the mycelium state with eight days of growth were placed in the center of the plates. Subsequently, the plates were incubated at room temperature (approximately 24° C.) for eight days (FIG. 28). The assay was performed in a triplicate. Finally, the percentage of radial growth inhibition (PICR) of the phytopathogen was measured using the formula PICR=(R1−R2)/R1×100 (Ezziyyani et al., 2004b).

The 4 Bacillus species evaluated showed an antagonistic effect against anthracnose (C. gloeosporioides) above 92% and 91% inhibition for Bacillus pumilus, and B. amyloliquefaciens, respectively, and 95% and 97% inhibition for Bacillus lichiniformis and B. subtilis, respectively.

### Preparation of Aloe Vera-Based Formulations for the Growth and Production of Lipopeptides of the 4 Bacillus Species

### Composition of Aloe Vera Gel

An analysis of the composition of the crystals/gel of the Aloe vera plant used was carried out in order to know the types of carbon and mineral sources that the bacteria can use and metabolize (Table 5).

TABLE 5
Chemical components of the Aloe vera plant

| Composition | Compounds |
|---|---|
| Carbohydrates | Cellulose, Glactose, |
| | Glucose, Arabinose, peptic |
| | substances |
| Minerales | Calcium, Magnesium, |
| | Zinc, Sodium, Phospor, |
| | Copper, Iron, Chrome |
| Vitaminas | Folic acid, cholin, vitamin |
| | E, B3 B6, beta caroten |
| Organic compounds | Saponins, gibberellins, |
| | uric acid |
| Amino acids | Valine, Thyrosine, Lysin, |
| | Proline, Methionine, |
| | Alanina |

### Preparation of Formulations Based on Aloe Vera

The Aloe vera leaves were washed and cut into thin layers macerated in a commercial blender. They were filtered, and the gel was stored in glass jars at 4° C. Subsequently, two dilutions (1:6 and 1:8) of the Aloe vera concentrate were prepared in sterile distilled water, supplemented with Tween 20. The MOLP medium optimized for the production of lipopeptides was used as a control (Hsieh et al., 2008) (Table 6).

TABLE 6
Composition of the Aloe vera formulations used as a substrate

| Formulation | Composition per 1 liter |
|---|---|
| AV 1 (1:6) | 400 ml of concentrate + 600 ml of water + 0.5 |
| | ml Tween 20 |
| AV 2 (1:8) | 600 ml of concentrate + 400 ml of water + 0.5 |
| | ml Tween 20 |
| Medium MOLP | Casein peptone 30 g/L, sucrose 20 g/L, yeast |
| (control) | extract 7 g/L, KH2PO4 1.9 g/L, MgSO₄ |
| | 0.45 g/L, citric acid 10 mg/L, FeCl₃•6H₂O |
| | 0.005 mg/L, MnSO₄ 3.6 mg/L. pH = 7 |

### Preparation of Bacterial Consortium Used in the Formulations

A pre-inoculum was prepared in 200 milliliters of LB broth for each one of the bacteria. They were incubated for 48 hours with shaking at 26° C. Later each pre-inoculum was centrifuged at 5,000 rpm for 10 minutes. The pellet of each bacterium was mixed in 100 mL of sterile saline solution (0.8%) in a 1:1 ratio and mixed for 10 minutes in an orbital shaker to homogenize the cells of each bacterium, later, in 1 liter of each aloe vera formulation, the concentration of the consortium was adjusted to 3.2×10⁹ CFU/mL using tube 5 of the McFarland scale, according to the methodology of Hsieh et al. (2008).

### Evaluation of the Production of Lipopeptides in 2 Formulations of Aloe Vera as a Substrate

The concentrations of lipopeptides were evaluated in the two formulations and in the control culture medium described above, which contained 2 mL of the bacterial consortium at a concentration of 3.2×10⁹ CFU/mL. Treatments with three replicates were placed in an orbital shaker at 120 rpm at 30° C. and incubated for 72 h.

The extraction and detection of the lipopeptides produced in vitro were carried out by collecting 12 mL samples from each treatment. Later they were centrifuged for 10 min at 2,300 rpm to transfer 10 mL of the supernatant through C18 solid phase extraction cartridges (Grace Maxi-Clean™ SPE 300 mg, Alltech Associates Inc.), previously rinsed with 20 mL of methanol and 15 mL of MilliQ water (ultrapure water). Following this, the lipopeptides adhered to the column were released by passing 1 mL of methanol through the cartridge and transferred to 1.5 mL microcentrifuge tubes. The samples were centrifuged again for 10 min at 2,300 rpm to transfer 300 μL to a unique tube for ultra-performance liquid chromatography-UPLC (Waters Acquity H-class) analysis, following the methodology of Montero et al. (2016).

Results of Lipopeptide Production

According to the results, only three polypeptides produced by the microbial consortium (Surfactin, Fengicin, and Iturin) were detected. In the control medium (MOLP), the production of the three compounds was obtained in the highest quantities, which confirms the antagonistic capacity of the bacteria of the Bacillus genus through the production of secondary metabolites such as polypeptides. The formulations with aloe vera drastically affected the consortium's growth, decreasing its concentration. Therefore, it also affected the production of polypeptides (Table 7). Iturin was not detected in the AV1 formulation with aloe vera (1:6 dilution), and the fengycin molecule was not detected in the AV2 formulation (1:8 dilution). The values found for the other polypeptides are not significant.

TABLE 7
Production of lipopeptides of the microbial consortium in 2 aloe vera formulations

| | Consortium | | | |
|---|---|---|---|---|
| | concentration | Lipopéptidos μ/mL | | |
| Formulation | (CFU/mL) | Surfactin | Fengycin | Iturin |
| AV1 | 1.5 × 10⁴ | 0.30 ± 0.01 | 0.20 ± 0.01 | ND |
| AV2 | 1.2 × 10⁶ | 0.03 ± 0.001 | ND | 0.20 ± 0.01 |
| MOLP | 4.1 × 10⁹ | 9.45 ± 0.1 | 14.39 ± 0.1 | 10.36 ± 0.2 |

### Conclusions

Bacillus pumilus bacteria, B. amyloliquefaciens, Bacillus lichiniformis, and B. subtilis are biological control agents of phytopathogenic organisms.

The four bacillus bacterial species can be used as consortium inoculums to increase their antagonistic activity against pathogens. The four species of the genus Bacillus can produce polypeptides in the control medium (MOLP). Lipopeptides produced by strains of the genus Bacillus represent promising and sustainable strategy for protection against pathogens. These compounds allow the reduction of synthetic fungicides and their harmful effects on the environment and health. However, the formulations with aloe vera did not work because they did not have the adequate nutritional composition for the growth of the bacteria. The lack of nitrogen sources in the aloe vera formulations is believed to have impaired the carbon to nitrogen (C/N) ratio being necessary for cell growth. Carbon sources such as arabinose and galactose present in aloe vera gel could negatively affect the load and survival of microbial cells. If the bacteria do not reach their exponential phase, the biosynthesis of secondary compounds, such as polypeptides, is affected.

### Example 3

The currnet invention relates to a bacterial consortium comprising three different strains of commercially available lactic acid bacteria, namely, Lacticaseibacillus casei, Lacticaseibacillus paracasei, and Lacticaseibacillus rhamnosus; grown in 3 solutions of extracts of vegetable residues of tobacco plants, guaranteeing a concentration of 9×10⁸ CFU. Two (2) dilutions and the botanical concentrate was used to evaluate the growth and cell viability of the bacteria that ensure the efficiency of the formulation in the control of the development of pathogens in fruits.

### Laboratory Tests

### Preparation of Individual Pre-Inocula of the 3 Lacticaseibacillus Bacteria

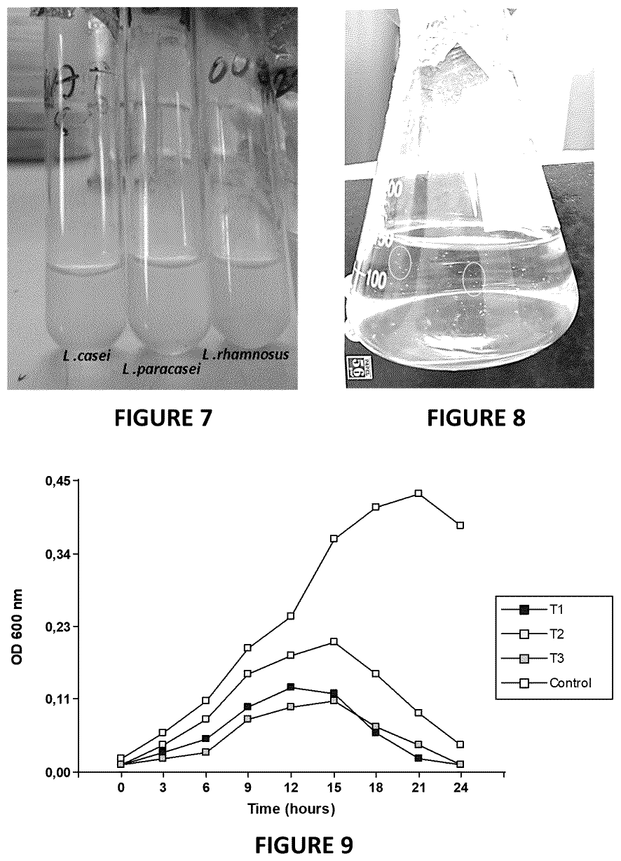

The stock cultures of each of the bacteria stored at −20° C. were activated using the platinum loop extension technique in Petri dishes with MRS-Scharlab agar culture medium following the methodology of Chang et al., (2010) and incubated at 37° C. between 48 and 60 hours. Subsequently, a pre-inoculum was made in 10 mL of 0.85% saline solution from pure colonies until reaching a concentration of 9×10⁸ CFU/mL corresponding to tube No. 2 of the Macfarland scale (FIG. 7).

### Preparation of Microbial Consortium

As a second step, 5 mL of each pre-inoculum was mixed in 15 mL of saline solution (1:1 ratio) to form the bacterial consortium, adjusted to a concentration of approximately 9×10⁹ CFU/mL, which corresponds to tube No. 3 of the scale of Macfarland, subsequently, the consortium was incubated for 10 minutes with shaking at 100 rpm to homogenize the cell suspension.

### Processing of Tobacco Residues

According to the bibliography, Xinda et al., (2021), tobacco plant residues (leaf veins, roots, and stems) have the following chemical composition:

TABLE 8
Chemical composition of tobacco plant
residues (Xinda et al., (2021))

| | Percent concentration/500 g |
|---|---|
| Components | of fresh tissue |
| Reducing sugars | 25.58 |
| Organic acids | 16.14 |
| proteins | 8 |
| Mineral substances | 14.25 |
| (K, Ca, Mg, Zinc) | |
| Nicotine | 1.04 |
| alkaloids | 5.23 |

The tobacco extracts used as vehicles in the formulation (concentrate and two dilutions) were prepared by macerating approximately 1 kg of plant residues from the tobacco plant in 1 liter of distilled water (1:1), subsequently filtered through an aluminum strainer to recover only the aqueous part. Table 9 shows the composition of the tobacco formulations. A solution of MRS-Scharlab medium was used as a control.

TABLE 9
Composition of the tobacco formulations used

| Formulations | Composition |
|---|---|
| T1 Concentrate 100% | 50 mL extract + 1 mL Ethylhexyl glycerin + |
| | 0.5 mL NaCl (0.1M) |
| T2 Dilution 50% | 25 mL extract + 25 mL H₂O + 1 mL Ethylhexyl |
| | glycerin + 0.5 mL NaCl (0.1M) |
| T3Dilution 25% | 12.5 mL extract + 37.5 25 mL H₂O + 1 mL |
| | Ethylhexyl glycerin + 0.5 mL NaCl (0.1M) |
| T4 Medium | 68.25 g of the powder in 1 liter of water |
| Commercial control | (following the manufacturer's instructions) |
| MRS -Scharlab | |

The pH of the formulations was adjusted to 6.8±0.1 and sterilized (autoclaved) at 120° C., 1 atm pressure for 20 minutes.

### Growth Kinetics of the Microbial Consortium in Formulations Based on Tobacco Extracts

An aliquot of 5 mL of the microbial consortium was inoculated in 50 mL of the prepared formulations, and in the control, subsequently, the absorbance at 600 nm was measured to have time 0, and then each formulation was incubated at 30° C. in a skaker at 130 rpm., a 1 ml aliquot was taken every 3 hours, and the absorbance was measured at 600 nm until the microbial consortium reached the stationary phase in each formulation. This procedure was performed in duplicate on each formulation, and the absorbance at 600 nm was plotted as a function of time.

Results of the growth of the bacterial consortium in three formulations with tobacco extracts and the MRS control medium.

The kinetics of bacterial growth consists of population growth by doubling the bacterial cells until the depletion of nutrients.

As shown in FIG. 9, the cell death phase in the three tobacco extract formulations was faster, at 12 hours for the concentrated formulation and with 25% and approximately 15 hours with the tobacco extract formulation. Tobacco at 50%, which indicates that the energy sources provided by the formulations were not enough for the cells to double and the population to increase, until reaching the exponential phase of growth with an optical density of 0.40-0.44, as can be seen in the control medium.

These results may be atributed to wild tobacco plants being capable of synthesizing a great diversity of secondary metabolites that are further activated by the maceration and incubation process, affecting the viability of the microbial consortium, decreasing the cell population and therefore Therefore, the consortium failed to reach the exponential phase since microbial growth is dependent on the availability of the substrate, especially carbon sources.

### Conclusions

The botanical extracts from tobacco waste do not provide the carbon and energy sources necessary for the incredible growth and multiplication of the bacterial cells of the consortium. Therefore, it is recommended to use other plant extracts that guarantee the minimum requirements necessary for the growth of bacteria. The three species of Lacticaseibacillus are compatible with each other and, therefore, can be used in consortia since they grew perfectly in the control medium.

### Example 4

The following composition is presented, which comprises a microbial consortium with stability and growth-promoting and biological control activity with Chirimola (Annona cherimola) plant extracts. Said composition can contain at least one secondary component and/or inert substances. The methodologies used for the biochemical characterization of the consortium are described;

The consortium is made up of 11 bacteria from 6 different genera: (i) Bacillus: Bacillus subtilis Q135, Bacillus amyloliquefaciens, Bacillus pumilus, and Bacillus licheniformis; (ii) Lacticaseibacillus: Lacticaseibacillus casei, Lacticaseibacillus paracasei, and Lacticaseibacillus rhamnosus; (iii) Latilactobacillus: Latilactobacillus sakei, (iv) Lactiplantibacillus: Lactiplantibacillus plantarum, (v) Lactococcus: Lactococcus lactis, and (iv) Limosilactobacillus: Limosilactobacillus fermentum.

### Laboratory Tests

### Compatibility Tests Between Bacteria

From the pure cultures of the 11 strains stored at −20° C. in nutrient broth supplemented with 40% glycerol, the procedure was carried out as described below.

Petri dishes containing LB nutrient agar medium were marked, dividing the plate into 11 equal parts.

Subsequently, the microorganisms were sowed using the exhaustion method (sowing on the surface), seeding a colony of each bacterium on each side. The test was performed in triplicate.

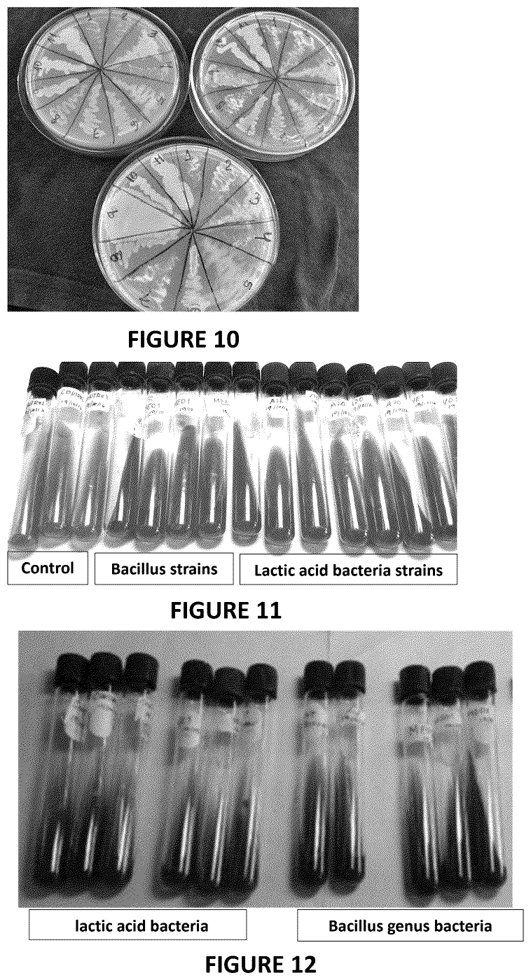

Then, they were incubated at 37° C. for 24 hours until growth was observed (FIG. 10).

As a result of these in vitro tests, it was concluded that the bacteria evaluated had presented a high degree of compatibility with each other; therefore, it is possible to include them in the same formulation and propagate them together in formulations.

### Biochemical Characterization

### Urea Hydrolysis Test

Urease is an essential microbial enzyme breaking organic compounds (Gunsalus & Stanier, 1961). Bacterial enzymes are classified as constitutive or adaptive; urease is considered constitutive because it is synthesized by certain bacteria regardless of the presence or absence of urea as a substrate (Burrows & Moulder, 1968). This test determines the ability of bacteria to split urea, forming two ammonium molecules by the action of the urease enzyme, producing a fuchsia color change in the medium. The bacteria were inoculated in a urea medium and incubated at 28° C. for five days for the test.

TABLE 10
Composition of the medium Christensen medium (Urea agar base)

| Composition | Concentration (g/L) |
|---|---|
| Tryptone | 1.0 |
| Glucose | 1.0 |
| Sodium chloride | 5.0 |
| Monopotassium phosphate | 2.0 |
| Phenol red | 0.012 |
| Agar | 15.0 |
| Final pH | 6.8 ± 0.2 |

In this test, the 11 strains evaluated were positive since a color change was detected in the urea-agar-base medium from red to fuchsia (Bailón, 2003) (FIG. 11).

### Citrate Agar Test

In Simmons Citrate Agar culture medium, mono ammonium phosphate is the only nitrogen source, and sodium citrate is the only carbon source (Table 11). Both components are necessary for bacterial development. In FIG. 12 shown are that the result obtained after growing the 11 bacteria in Simmons Citrate Agar culture wedges. This test showed a positive response for the four bacteria of the Bacillus genus (FIG. 12) since a color change from green to blue was observed, thus, indicating that these strains can metabolize sodium citrate as a carbon source. Lactic acid bacteria did not change the coloration of the medium.

TABLE 11
Composition of Simmons Citrate Agar Medium

| Composition | Concentration (g/L) |
|---|---|
| Sodium citrate | 2.0 |
| NaCl | 5.0 |
| Dipotassium phosphate | 1.0 |
| Monoammonium phosphate | 1.0 |
| MgSO₄ | 0.2 |
| Bromothymol blue | 0.08 |
| Agar | 15 |
| Final pH | 6.3 |

The production of low molecular weight organic acids by bacteria is one of the most widely known mechanisms of soil phosphate dissolution, which makes phosphorus (P) available for plant nutrition. The action of organic acids in mineral dissolution can be attributed to the fact that they lower the pH and, even more, to the formation of stable complexes with Ca²⁺, Mg²⁺, Fe³⁺, and Al³⁺. Organic acids have been shown to reduce phosphate precipitation by iron and aluminum (Shippers, 1987; Moghimi & Tate, 1978).

### Nitrate Reduction

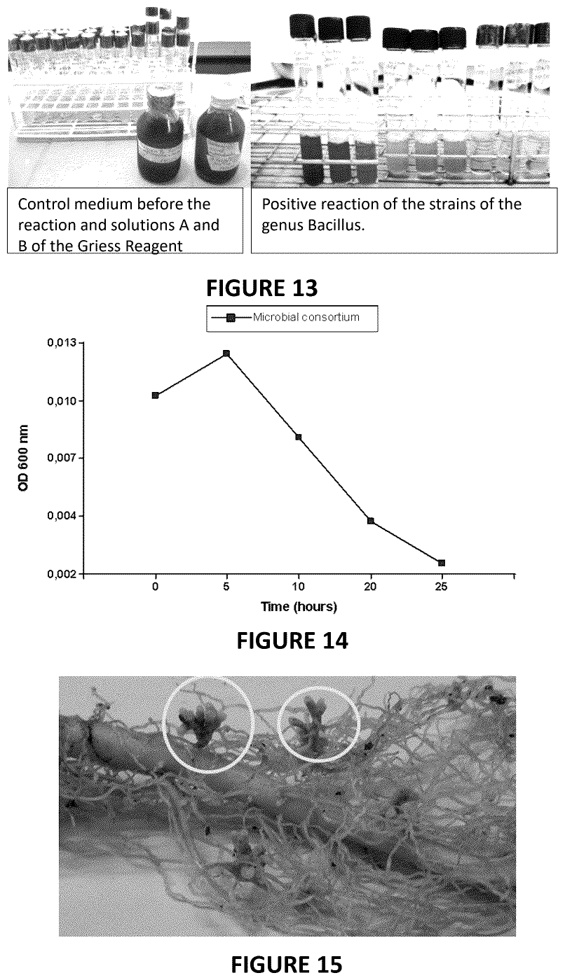

Some bacteria can reduce nitrates to nitrites, a such characteristic it is used for the identification and differentiation of many bacterial species (Bailón, 2003). Eleven (11) bacteria were inoculated in Nitrates broth (Table 12) contained in test tubes and incubated at 28° C. for eight days with shaking at 100 rpm. After this time, the reducing capacity of nitrates was evaluated by adding three drops of α-naphthylamine and three drops of sulfanilic acid under an extraction hood, observing the color change from yellow to red as a positive result, according to the MacFaddin (2000) methodology (FIG. 13).

TABLE 12
Composition of the nitrate broth medium

| Composition | Concentration (g/L) |
|---|---|
| Meat extract | 3.0 |
| Peptone | 5.0 |
| Potassium nitrate | 1.0 |

Griess reagent: Sol A and Sol B

This test is carried out to determine the ability of bacteria to reduce nitrates to nitrites, and the enzyme nitrate-reductase carries it out, Positive bacteria indicate that they can use nitrate as a source of energy.

TABLE 13
Summary of the biochemical characterization of the 11 bacteria

| | test | | |
|---|---|---|---|
| | Urea | Simmons | Nitrate |
| bacteria | hydrolysis | citrate | reduction |
| Bacillus subtilis Q135 | + | + | + |
| Bacillus amyloliquefaciens | + | + | + |
| Bacillus pumilus | + | + | + |
| Bacillus licheniformis | + | + | + |
| Lacticaseibacillus casei | + | − | − |
| Lacticaseibacillus paracasei | + | − | − |
| Lacticaseibacillus | + | − | − |
| rhamnosus | | | |
| Latilactobacillus sakei | + | − | − |
| Lactiplantibacillus | + | − | − |
| plantarum | | | |
| Lactococcus lactis | + | + | − |
| Limosilactobacillus | + | − | − |
| fermentum. | | | |

+, Positive result;
− Negative result

### Process of Elaboration of Microbial Consortium in Extracts of Plant

For the formulation, 500 g of fresh chirimoya leaves (Annona cherimola) were used, which were mixed in 950 mL of water and 50 mL of 5% sodium hydroxide. The mixture was left to digest for one hour and then passed through a metal strainer to remove the remains of plant tissue. The microbial consortium was prepared in saline solution at 1×10⁹ of all bacteria. Subsequently, other compounds, such as those shown in Table 14, were added,

TABLE 14
Composition of the formulation based on extracts
of cherimoya (Annona cherimola)

| Formulation | Concentration and Composition |
|---|---|
| Active ingredient | Microbial Consortium 20% (1 × 10⁹) |
| substrate/vehicle | 30% Chirimola plant extract |
| | (after digestion with NaOH) |
| emulsifier | 1.5% |
| stabilizer | 2.0% |
| diluent | 46.5 |
| Total | 100% |

Survival of the Bacterial Inoculum in the Formulation after 24 Hours

The bacterial growth curve results from the graphic representation of the periodic determination of the number of viable cells per milliliter in the formulation. Since the beneficial effect of the consortium as a probiotic depends on its amount, the growth of the consortium in the formulation was analyzed after 5, 10, 20, and 25 hours (FIG. 14).

According to FIG. 14, it can be seen that the microbial consortium did not develop the number of cells necessary to multiply. Subsequently, it was determined that these results are because the chirimoya extracts and sodium hydroxide produced cell lysis in most of the bacteria, especially of the Bacillus genus, since the formulation was alkalinized after the incubation time. The consortia reach a pH of 9.6, which considerably affects the osmotic pressure of the bacterial cells. On the other hand, the formation of Bacillus endospores was not observed.

### Conclusions

The survival of probiotic microorganisms depends on various factors such as the specific characteristics of the genus and species, the matrix in which it is found (food, excipient, etc.) since each product has different pH characteristics, and available nutrients, as well such as environmental storage conditions such as temperature, humidity, type of packaging, presence of light.

### Example 5

Further provided herein is a consortium made up of bacterial strains belonging to different genera, such as Bacillus, Lactobacillus, and Azospirillum, e.g., A. brasilense KF2212 isolated from the corn crop (Zea Mays). The multispecies consortium can be applied to the plants' soil, seeds, roots, leaves, and fruits. The formulation matrix includes citrus extracts as well as stabilizers and/or preservatives.

### Laboratory Tests

Isolation of Azospirillum Sp Strains from Corn Crops

Maize plants (Zea mays) were collected and taken to the laboratory, where the plant roots were gently washed with distilled water to preserve soil adhering to the roots. Subsequently, the rhizosphere samples were seeded in flasks containing nitrogen-free broth (NFb) using malate as a carbon source (Döbereiner et al., 1976). They were then incubated at 33° C. in a culture oven until a white, thick, wavy film was developed below the surface. Subsequently, 1 mL of this film was extracted to carry out serial dilutions to the tenth with 0.8% saline solution until reaching a dilution of 10-4 and 10-5 of the original culture. Subsequently, they were inoculated in a selective Congo red malic acid agar medium at 33° C. for 96 h. Isolated scarlet red colonies with abundant growth, dry consistency, rough surface, and irregular edge were considered positive samples, as observed in FIG. 1 (Rodriguez-Caceres, 1982).

### Molecular Identification of Azospirillum Sp Isolate

Comparing the sequences of the 16S rRNA (or of the genes that encode it) makes it possible to establish the phylogenetic relationships between prokaryotic organisms. This fact has enormously impacted bacterial taxonomy, giving rise to the current classification system and allowing the rapid and accurate classification of bacteria. The isolates were identified by PCR amplification of the 16S rRNA, its partial sequence (with reading in two directions), and sequence analysis. First, DNA extraction was carried out following the methodology of Cassán et al. (2014).

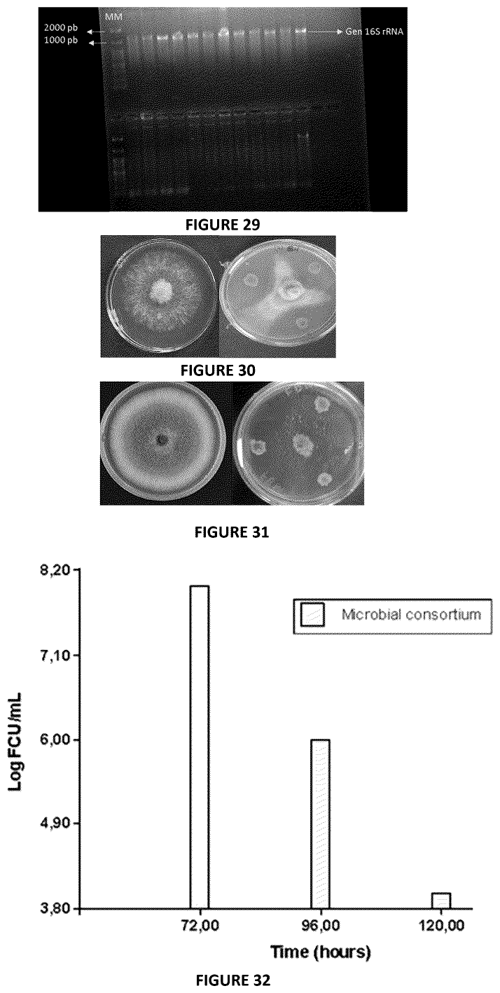

Once the integrity of the rRNA was confirmed, PCR was performed with the specific primers 27f (5′-GAGAGTTTGATCCTGGCTCAG-3′; SEQ ID NO: 3) and 149r (5′-CTACGGCTACCTTGTTACGA-3′; SEQ ID NO: 4) that amplify a fragment of the 16S rDNA gene. The reaction mix for a final volume of 25 μL was: 2.5 μL 10× Taq Pol Buffer, 2 μL 25 mM MgCl₂, 1 μL 10 mM dNTPs, 1 μL 10 μM 27f+1492r primer mix, 0.2 μL 500 U Taq Polymerase (Thermo Scientific), 2 μL template DNA, and 15.3 μL sterile miliQ water. The parameters for the PCR were: one cycle at 95° C. (5 min), 55° C. (1 min), and 72° C. (2 min); 30 cycles at 94° C. (45 s), 50° C. (2 min) and 72° C. (50 s) and a final cycle at 72° C. for 10 min. The reaction results (5 μL) were analyzed on a 1.5% agarose gel, using 0.5× Tris-boric acid-EDTA (TBE) buffer as the running buffer. To visualize the amplified products, 1 μl of GelRed 10,000× (Biotium) was added for every 5 μl sample. The gel was visualized through UV light (320 nm) with a Vilber Lourmat TFX-20M transilluminator (FIG. 29).

The molecular studies confirmed the results obtained by traditional microbiological methods (such as selective media and morphologies) that located the isolated strain of Azospirillum sp within the Azospirillum brasilense species and were identified as Azospirillum brasilense KF2212.

The assignment of taxonomic categories (species or strain) of microorganisms was for many years based on the distinction of morphological, biochemical, and/or antigenic characteristics, and in the best of cases, on physiological aspects detectable by chemical or biochemical analysis; however, with the development of genomic techniques, some chemical and physicochemical characteristics of nucleic acids were first considered. Later, sequence comparisons of relatively short fragments or a pair of genes were made (Valenzuela et al., 2015).

### Preparation of Pre-Inoculum of the Bacteria to Form the Consortium

The cultures of the Bacillus and Azospirillum strains were prepared to start from cultures preserved in Petri dishes with Nutrient Agar (AN) medium, from which a sample of each one was taken and seeded by exhaustion in the same medium and incubated at 28° C. for three days to obtain pure and fresh colonies. Subsequently, a pure colony of each strain was seeded in an NA liquid medium and incubated at 28° C. with shaking at 100 rpm for three days.

To prepare the pre-inoculum of the lactic acid strains, a pure colony of each species was taken and cultured in MRS medium broth (Agar-Scharlab) to be incubated at 26° C. with shaking at 100 rpm for three days.

Subsequently, after all the strains had grown in their respective media, the microbial consortium was prepared; for this purpose, 5 mL of each bacterium was centrifuged at 5,000 rpm for 10 minutes, and the pellet was resuspended in 5 mL of sterile distilled water, followed by the concentration of each bacterial suspension was adjusted for turbidity in 10 mL of 0.8% saline solution using tube 3 of the McFarland scale, which corresponds to a concentration of 9×108 CFU. Finally, all the bacteria were mixed in a 1:1 ratio and stirred for 10 min to homogenize the cell suspension.

### Isolation of Phytopathogenic Fungi

The phytopathogenic fungus Botrytis cinerea was isolated from strawberry plants in greenhouses, plants with damaged fruits were selected, showing a brown cover with a spore carpet texture, a peculiar symptom of the presence of Botrytis in that strawberry, and for the isolation of Colletotrichum gloeosporioides (anthracnose) was isolated from stored orange fruits. Fragments of infected tissue were selected as inoculum sources and established under aseptic conditions in Petri dishes with commercial Potato-Dextrose (PDA) agar culture medium and incubated in a growth chamber at 25° C. and 80% relative humidity for eight days.

### In Vitro Confrontation Assays

For the bioassay, a commercial PDA culture medium was used. One liter of the medium was prepared following the manufacturer's instructions and sterilized for 20 min in an autoclave at 120° C. and 15 lb of pressure. Subsequently, the medium was poured into sterile Petri dishes and solidified at room temperature for 24 h. The following day, the plates were divided into three equal parts. The microbial consortium was inoculated with 20 μl at the ends of the Petri dishes and then incubated for 24 hours at 26° C. Finally, a PDA disc was deposited with the inoculum of B. cinerea and C. gloeosporioides obtained with a 3.5 mm diameter sterile punch placed inverted in the center of the box. Petri dishes were incubated at 26±2° C. inside a growth chamber. From the seeding of the fungus in the Petri dish and for eight days, the diameter of the mycelium was measured with a digital vernier.

### Results of In Vitro Confrontations

The results obtained in the micellar growth inhibition tests showed significant differences, confirming the evaluated microbial consortium's antagonistic effect on the micellar growth of B. cinerea, inhibiting more than 85% compared to the control (FIG. 30). In the same way, the pathogen C. gloeosporioides showed the highest percentage of inhibition at 93% (FIG. 31).

Cellulases, chitinases enzymes, and volatile compounds lyse the membrane of phytopathogenic fungi, affecting their growth and development. These characteristics have been described as mechanisms involved in the biological control of pathogens by competition for nutrients and resources since it allows bacteria with antagonistic action to access and colonize a habitat displacing or limiting the colonization of other microorganisms.

Formulation of the microbial consortium comprised of strains of Bacillus, Lactobacillus, and Azospirillum brasilense KF 2212 in a matrix of citrus fruits.

The vehicle tested in this formulation is composed of citrus fruit extracts, basically, Citrus Aurantium Amara (bitter orange) at 2.2% and Citrus reticulata (mandarin) at 1.5%, essential oils at 1.5%, supplemented with Ethylhexyl glycerin at 2.5% and 92.5% of inert substances and diluent (saline solution 0.8%). The pH of the formulation was adjusted to 6.5 with 1 N NaOH. These botanical extracts can be exploited as innovative substrates to protect and increase the stability of bacterial inoculums in biological control products. The formulation was evaluated at three culture times, 72, 96, and 120 hours after inoculation with the microbial consortium. At each moment of the culture, the cell viability of the consortium was determined by the formation of colonies (CFU) by the drop method and plating in Petri dishes with nutrient agar.

### Results

It is observed that after 72 and 96 hours of culture, the largest populations of the consortium were found (1.7×10⁹ and 6.3×10⁸ CFU/mL), which indicates that the matrix provides the necessary nutrients for cell multiplication and that despite the time elapsed, there were no changes in the pH of the formulation that could affect the growth of the bacteria. After 120 hours of culture, there was a drop in cell counts and decreased cell viability, indicating that the consortium would enter the cell death phase (FIG. 32).

### Conclusions

The isolation of the bacterium Azospirillum brasilense KF 2212 completes the microbial consortium evaluated in other formulations. This new bacterium fixes nitrogen and produces phytohormones that favor plant growth. The consortium presented a high potential as a biological control agent for phytopathogens. The compatibility of the different bacterial species that comprise the consortium was determined. It is a liquid formulation with inert substances, diluents, and citrus extracts. The formulation can be applied to the soil, roots, leaves, fruits, and other plant surfaces.

### Example 6

Further, it was shown that the disclosed formulation provides a synergistic effect between different microbial species, plant extracts of Brassica oleraceae var-Capitata, and extracts of fermented citrus fruits. The microbial consortium of this formulation combines the antibiosis effect of 4 Bacillus species with the ability to make nutrients available from the Azospirillum brasilense and Rhizobium etli AM001 strains, the latter been isolated and characterized by fixing nitrogen and solubilizing phosphorus. In addition, it also includes different species of probiotics, such as Lactobacillus, that help improve plant nutrient absorption and increase the production of plant growth hormones, such as auxins and cytokinins. All strains are classified as beneficial and coexist with each other without being inhibited.

### Laboratory Tests

### Isolation and Identification of Rhizobium sp Strains

Three plants in alfalfa (Medicago sativa) were collected at equidistant points of approximately 1.5 meters, sampling was performed in triplicate, and nodular root samples were taken to the laboratory for processing. Next, the roots were carefully washed with distilled water to avoid detaching the nodules (FIG. 15).

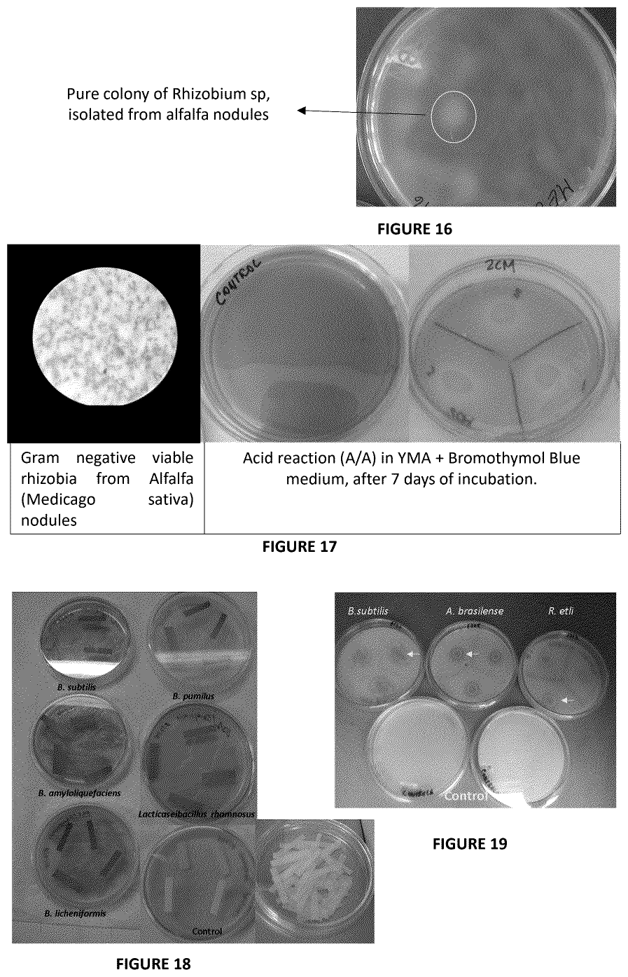

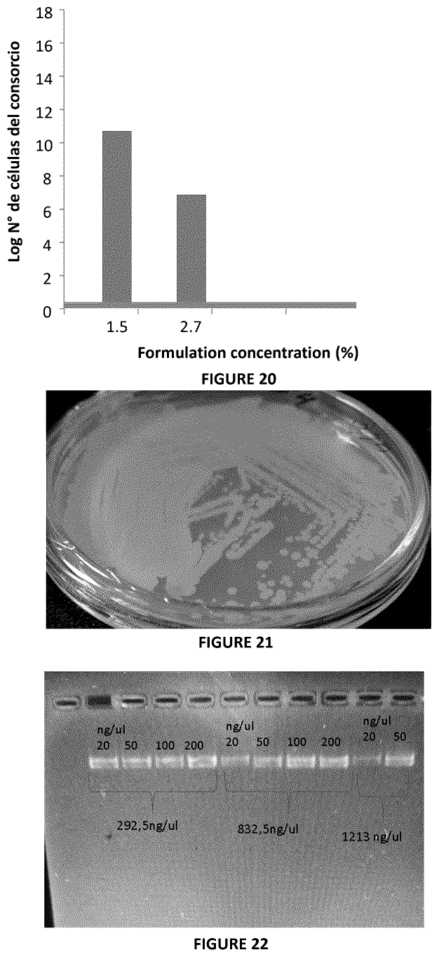

Following the methodology of Matos et al. (2007), the nodules were disinfected by successive immersion in 70% alcohol for one minute and 2.5% sodium hypochlorite for four minutes, followed by 7 to 8 rinses with sterile distilled water., then one nodule per tube was placed with 3 mL of sterile distilled water and macerated with a sterile glass rod until a cloudy solution was obtained to seed 10 μl of the solution by depletion in boxes of YMA medium following the methodology of Vincent, 1975.; Ferrera Cerrato et al., (1993). (K₂HPO₄ 0.655 g, MgSO₄·7H₂O 0.2 g, NaCl 0.1 g, Mannitol 2.5 g, Sucrose 7.5 g, Yeast extract 0.5 g and agar 15 g. plus 1 mL of Red solution Congo (1 g of RC in 400 mL of distilled water) pH of the medium 6.5-6.8 Subsequently, they were incubated at 30° C. until pure colonies of Rhizobium sp were obtained, as shown in FIG. 16.

### Morphological and Metabolic Identification of Rhizobium sp.

The colonies obtained in isolation in the YMA medium were selected. The selection criteria were based on the characteristics proposed by Martínez et al., 2005; as Gram-negative colonies; pH reaction test in YMA medium+Bromothymol blue. (In addition to morphological aspects such as shape, border, color, and appearance of the colonies). Through molecular biology tests, the strain was identified as Rhizobium etli AM001.

Characterization of the Microbial Consortium Based on Secondary Metabolites with Antagonistic Action (Hydrocyanic Acid)

The microbial consortium comprises the species mentioned in Table 15, and the concentrations used in the HCN production tests are also shown.

TABLE 15
Composition and concentrations of the bacteria that
make up the microbial consortium of the formulation.

| Bacterial inoculum | Concentration (CFU/mL⁻¹) |
|---|---|
| Bacillus subtilis Q135 | 1 × 10⁸ |
| Bacillus amyloliquefaciens | 1 × 10⁵ |
| Bacillus licheniformis | 1 × 10⁴ |
| Bacillus pumilus | 2 × 10² |
| Lacticaseibacillus casei | 2 × 10² |
| Lacticaseibacillus paracasei | 2 × 10⁴ |
| Lacticaseibacillus rhamnosus | 2 × 10⁴ |
| Latilactobacillus sakei | 2 × 10⁵ |
| Lactiplantibacillus plantarum | 2 × 10⁵ |
| Lactococcus lactis | 2 × 10² |
| Limosilactobacillus fermentum | 1 × 10⁶ |
| Azospirillum brasilense KF2212 | 1 × 10⁷ |
| Rhizobium etli AM001 | 1 × 10⁸ |

HCN Production Test (Volatile Compound) of Bacteria that Make Up the Microbial Consortium of the Formulation

The different species of bacteria were cultivated in the King B agar medium modified with glycine (4.4 g·L⁻¹). A 4 cm long strip of Whatman no. 1, previously soaked with a sterile solution of 1% picric acid in 10% sodium carbonate (filter paper and picric acid were sterilized separately). The plates were sealed with Parafilm since HCN is volatile and incubated at 28° C. for 48 hours. Three boxes per bacterium were planted. The orange color on the filter paper strips indicates positive results for HCN production (Ahmad et al., 2008). The 4 strains of the Bacillus genus; B. substilis, B. amyloliquefaciens, B. licheniformis, B. pumilus, and Lacticaseibacillus rhamnosus were positive for HCN production (FIG. 17).

### Phosphate Solubilization Test

The phosphate solubilization test was performed according to Malboobi et al. (2009). The Petri dishes were divided into three equal parts and 10 μl of the bacteria were inoculated on solid Pikovskaya medium (15 g agar, 10 g glucose, 5 g Ca₃ (PO₄)2, 0.5 g yeast extract, 0.5 g of (NH₄)₂SO₄, 0.2 g of KCl, 0.1 g of MgSO₄7H₂O, 0.1 mg MnSO₄₂H₂O, 0.1 mg of FeSO₄ in 1,000 mL of distilled water) and incubated at 30° C. for seven days. The development of a clear zone (halo) around the colony indicated inorganic phosphate solubilization.

### Nitrogen Fixation Test

Determining the probable biological fixation of nitrogen (FBN), the strains were shown in a semisolid medium FBN (Naher et al., 2009) free of nitrogen. This medium indicates the ability of the inoculated microorganisms to grow in the absence of mineral nitrogen and the presence of malic acid as the only carbon source. All the strains grew in FBN medium, indicating that they fixed nitrogen.

Evaluation of the Growth of the Microbial Consortium in Two Concentrations of Cabbage Extracts (Brassica Oleraceae Var. Capitata)

Cabbage is a plant that produces an enzyme called myrosinase that helps form glucosinolates, which are natural plant products with antifungal and anticancer properties. This plant has a fumigant action on pathogenic fungi, a product of the volatile substances released during the biodecomposition of fresh matter. The preparation of the extracts consisted of placing 500 grams of fresh cabbage leaves in 1 liter of water with 1 ml of tween 20; the solution was left macerating for 24 hours at 5° C. Subsequently, it was liquefied, and the solution was centrifuged for 5 minutes at 4000 rpm. The supernatant was mixed with the citrus fruit extracts, adherents, preservatives, and the microbial consortium (Table 16).

TABLE 16

| Formulation | Concentration and Composition |
|---|---|
| Active ingredient | Microbial consortium 25% in saline solution |
| | (CFU 1 × 10⁹) |
| Substrate/vehicle | 1.5/2.7% |
| between Brassica | |
| and citrus fruits | |
| emulsifier | 1.0% |
| stabilizer | 1.5% |
| diluent | 71% for the concentration of 1.5% extracts. |
| | 69.8% for the concentration of 2.7% extracts. |
| Total | 100% |

In this assay, the cell growth and viability of the microbial consortium were evaluated and tested in 2 concentrations of the formulation, 1.5 and 2.7%, respectively (FIG. 18).

The population of the microbial consortium began with concentrations around 1012 CFU, losing three exponential units after 96 hours of fermentation, which could be caused by the low oxygen tension (O₂) and the instability in the pH of the formula since after that time, the solution became acidic.

### Conclusions

The consortium includes a tremendous microbial diversity represented by lactic acid bacteria, nitrogen-fixing bacteria, and phosphate solubilizers.

The functions related to the availability of nutrients increase photosynthetic activity and water absorption in plants due to better development of the root system.

The bacterial species that comprise the consortium used in this formulation as an active ingredient have great potential for biological control due to the production of secondary metabolites such as HCN (cyanogenesis activity).

The concentration of 1.5% in the formulation allowed the survival of the bacterial cells for a longer incubation time.

### Example 7

Further disclosed herein is a microbial composition with a significant impact on the growth and yield of crops through the biosynthesis of biologically active substances, which can act as inhibitors of phytopathogens through the efficient production of chelating compounds such as Siderophores, in addition to the production of phytohormones and solubilization of nutrients that contribute to stimulating growth and a better physiological state of the plant (e.g., to face any stress). The matrix surrounding this efficient microbial consortium is a mixture of alginate produced by the microorganism and commercial starch.

### Laboratory Tests

Isolation and Identification of a New Strain that Makes Up the Microbial Consortium of the Formula, Belonging to the Azotobacter Genus

For the isolation of bacteria of the Azotobacter genus, five wheat (Triticum durum) plants were randomly selected, and roots were collected by drilling 20 cm from the soil to collect approximately 200 grams of roots and rhizospheric soil, which were subsequently stored in plastic bags, and taken to the laboratory until further processing. Next, 1 g of the root was washed 3 to 4 times with running water and then macerated in 5 mL of sterile distilled water. The root extract was centrifuged at 3,000 rpm for 10 min, and an aliquot (100 μL) of the 10-4 and 10-5 decimal dilutions was used to inoculate solid NF agar plates for the growth of the strains. The plates were incubated at 30° C. for 24 to 72 hours. Subsequently, the colonies that showed morphological characteristics similar to Azotobacter were isolated, subcultured, and observed under a light microscope. The subculture was carried out 3 times until obtaining pure colonies, as shown in FIG. 19.

### Molecular Identification of Azotobacter Strain

DNA was extracted from a pure colony of the strain following the Chen and Kuo (1993) method and subsequently amplified using the Weisburg (1991) method. Polymerase chain reaction (PCR) was performed using two primers rd1 and fd1, with the following nucleotide sequences: 5′-AAGGAGGTGATCCAGCC-3′ (SEQ ID NO: 5) and 5′-GAGTTTGATCCTGGCTCAG-3′ (SEQ ID NO: 6), respectively. Each reaction volume was 25 μL and contained 1 μL of 50 μg genomic DNA, mix of 0.2 mM dNTP, 1.5 mM MgCl₂, 5 AL 10× Taq buffer, 1 U Taq DNA polymerase, 10 pmol of each primer and the remainder of the volume was adjusted with ultrapure water. Reactions were carried out in a thermocycler (Biometer). After denaturing at 95° C. for 2 min, samples were held for 30 cycles through the following temperature profile: denaturation at 94° C. for 30 s, annealing at 55° C. for 30 s, extension at 72° C. for two min. 2 min plus an additional cycle for chain elongation at 72° C. for 10 min. FIG. 22 shows the 16S gene of the Azotobacter strain amplified in an agarose gel and with different concentrations of DNA, evidencing the integrity of the DNA,

After purification of the 200 ng/μl bands that were selected as the best using the QUICK Kit (GENOMED), sequence comparison was performed via the Internet using the bioinformatics tool Local Type Sequence Alignment (BLAST) in the National Center for Biotechnology Information (NCBI) database. The strain was identified as Azotobacter vinelandii AS122.

Biocompounds Produced by Bacteria from the Microbial Consortium that Comprise the Formulation

The bacterial species and concentrations used in the metabolite biosynthesis tests are described in Table 17.

TABLE 17
Bacteria that make up the microbial consortium

| Strain | Concentration |
|---|---|
| Bacillus subtilis Q135 | 1 × 10⁸ |
| Bacillus amyloliquefaciens | 1 × 10⁵ |
| Bacillus licheniformis | 1 × 10⁴ |
| Lacticaseibacillus casei | 2 × 10² |
| Lacticaseibacillus paracasei | 2 × 10² |
| Lacticaseibacillus rhamnos | 2 × 10⁴ |
| Latilactobacillus sakei | 2 × 10⁴ |
| Lactiplantibacillus plantarum | 2 × 10⁵ |
| Lactococcus lactis | 2 × 10⁵ |
| Limosilactobacillus fermentum | 2 × 10² |
| Rhizobium etli AM001 | 1 × 10⁶ |
| Azospirillum brasilense | 1 × 10⁴ |
| KF2212 | |
| Azotobacter vinelandii AS122 | 1 × 10⁶ |

### Biosynthesis of Gibberellic Acid (GA3)

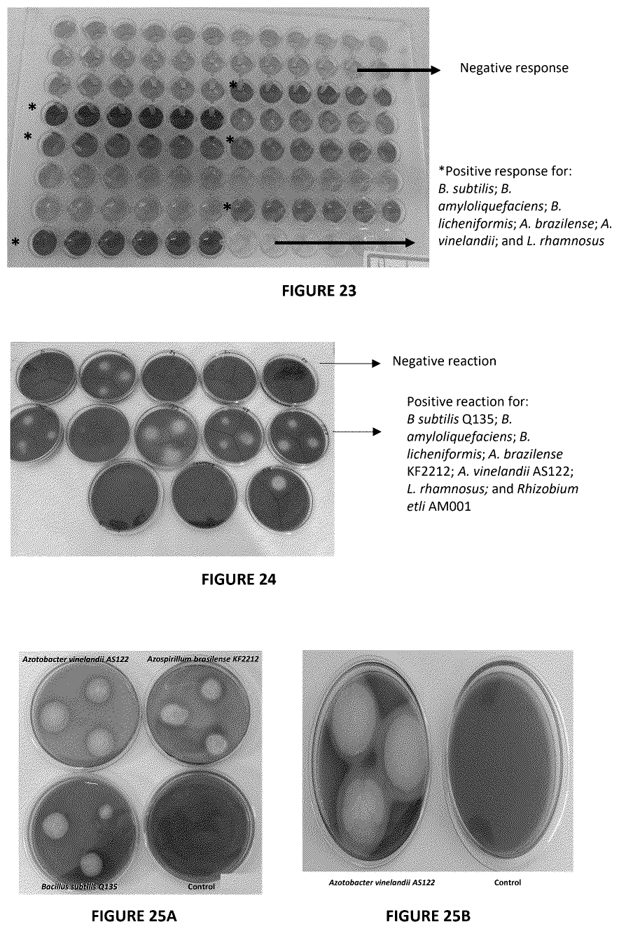

GA3 quantification was performed by the DNPH (2,4-Dinitrophenylhydrazine) method following the methodology of (Graham & Thomas, 1961) modified by Sagar (2017). The strains used for the microbial consortium were grown in falcon tubes with 10 mL of nutrient broth for 48 hours at 25° C. with shaking at 120 rpm for six days. Subsequently, the samples were centrifuged at 4,000 rpm for 10 min to eliminate cell biomass, and GA3 quantification was performed on the sixth day of incubation. For this, 2 mL of the supernatant was transferred to another tube and mixed with the same volume of ethyl acetate to extract the compound. The samples were mixed vigorously for 10 minutes and left to settle for 5 minutes. The ethyl acetate layer was then transferred to a new tube; this step was repeated three times with each sample. Subsequently, the ethyl acetate fractions were combined and evaporated at room temperature. The dried pellet of each bacterium was resuspended in 2 mL of absolute alcohol and mixed with 1 mL of 2,4-dinitrophenylhydrazine (1 mg/mL in concentrated HCL) and incubated at 100° C. for 5 min, then cooled with ice to being able to add 5 mL of 10% potassium hydroxide dissolved in absolute alcohol and let it stand until it turns brown. Finally, the contents were diluted 2:1 using sterile distilled water, and the absorbances were read at 430 nm in a microplate spectrophotometer (Thermo Scientific™ Multiskan™). The GA3 concentration was calculated from the regression equation of the standard curve of pure gibberellic acid (Sigma) prepared from 10 to 100 mg dissolved in absolute alcohol and expressed in μg/mL. GA3 production is positive if the solution turns yellow to brown/brown. Six times the volume of the same sample was placed (FIG. 23).

### Solubilization of Potassium

The Potassium solubilization test was performed following the Parmar and Sindhu (2013) methodology using Aleksandrov's modified culture medium (ADVm) containing: Glucose 10.0; g/L; 5.0 g/L (NH₄)₂SO₄; 0.5 g/L MgSO₄ 7H₂O; 1 0.5 g/L Yeast extract, and 0.5 ml bromocresol purple, pH was adjusted to 7.0. The medium has a purple color. Subsequently, ten microliters of each bacterium were cultured at the ends of the Petri dishes and incubated at 26° C. for five days until change in color was evident, e.g., indicative of positive bacteria.

### Production of Siderophores as a Secondary Metabolite for the Control of Phytopathogens

The ability to produce siderophores was evaluated using the Universal CAS (chrome azurol s) assay in a solid medium following Schwyn and Neiland's (1987) methodology. This method is the most widely used for detecting siderophores due to the ferrichromogenic complex that changes color due to the loss of iron. All the material used to prepare culture media and solutions was washed with alcoholic potash (0.5 N) to eliminate organic residues and avoid contamination. The CAS-agar medium was prepared to start from solutions A, and B. Solution A was prepared by adding 60.5 mg of chromium azurol s (CAS; Sigma-Aldrich 199532), 10 mL of a 10 mM ferric HCl solution with 1 mM of FeCl₃ 6H₂O and 72.9 mg of hexadecyltrimethylammonium bromide (HDTMA; Sigma-Aldrich H6269) dissolved in a final volume of 100 mL of distilled water.

The CAS was dissolved in approximately 40 mL of distilled water to add the iron solution and the HDTMA later. Finally, the solution was made up to a final volume of 100 mL with distilled water. Separately, as solution B, solid minimal medium 9 (MM9) was prepared (10 g/L glucose, 0.5 g/L NaCl, 0.3 g/L K₂PO₄, 0.1 g/L NH₄CH₃CO₂, 5.0 g/L, 30 g/L of MES) the pH of the medium was adjusted to 6.8 and 15 g/L of agar were added to make up to 900 mL with distilled water.

Both solutions were sterilized separately at 120° C. and 15 lb of pressure for 15 minutes. Once solutions A and B were sterile, they were allowed to cool to approximately 40° C. The 100 mL of the CAS solution was mixed with the 900 mL of MM9 to obtain 1 L of CAS-agar, acquiring the characteristic blue coloration of the medium. The medium was emptied into Petri dishes, and once solidified, the consortium bacteria were inoculated, placing 10 μl at the ends of the plates. Cultures were observed for 3 to 5 days and incubated at 30° C. Siderophore production was considered positive when observing a change from blue to yellow, orange, or purple depending on the type of siderophore present and the intensity of production (Pérez Miranda et al., 2007). Plates with solid CAS-agar medium without inoculation were used as controls.

### Encapsulation of the Microbial Consortium Using an Alginate Matrix Produced by Azotobacter Vinelandi Plus Starch

Encapsulation is the physicochemical process in which microorganisms are trapped in a semipermeable membrane, producing a capsule with a small diameter, where they float freely without affecting their biological activity (Medina & Huertas, 2012). Likewise, encapsulation contributes to the stability, bioavailability, and conservation of bioactive components and the viability of microorganisms (Lupo et al., 2012).

### Preparation of the Inoculum

Ten (10) mL were inoculated at 10⁸ CFU/mL concentrations for all the consortium bacteria in 90 mL of fresh LB liquid medium. The media were incubated for 48-72 hours at 28° C. with constant shaking at 120 rpm. After the incubation, an inoculum of 10 mL of each microorganism was taken and centrifuged for 5 minutes at 5,000 rpm. The supernatant was discarded and centrifuged again for two more minutes. Subsequently, 3.3 mL of the pellet obtained was taken by building the biomass destined for encapsulation following the methodology of Hernández et al. (2011).

### Preparation of Commercial Starch and Microbial Alginate Solutions

Stock solutions of 4% alginate and 15% starch were made and stored at room temperature until use. The bacterial alginate was obtained from a pure culture of Azotobacter vinelandii AS122, cultivated for 72 hours under both nutritional and thermal stress conditions to increase biofilm and alginate production. In order to establish the relationship of the matrices concerning the stability of the microorganisms, a concentration of a final volume of 10 mL of solution was started with the following relationships: Alginate matrix 2%-starch matrix 1.5% likewise, a volume of 3.3 ml corresponding to each microorganism and each polymer was taken for its subsequent encapsulation (Table 18).

TABLE 18

| | Microbial Starch-Alginate Relations | | |
|---|---|---|---|
| Formulation/Matrix | mL | (% m) | Stock |
| Starch | 3.3 mL | 1.5% | 15% |
| Alginate | 3.3 mL | 2% | 4% |
| Microbial consortium | 3.3 mL | — | — |
| (2.2 × 10⁹ CFU/mL) | | | |

All the species of the bacterial consortium were cultured and adjusted to 2.2×10⁹ CFU/mL individually. Later for encapsulation, 1 ml of each was mixed and incubated for 15 minutes at room temperature to homogenize the cell suspension.

### Encapsulation of Microorganisms in the Alginate-Starch Matrix

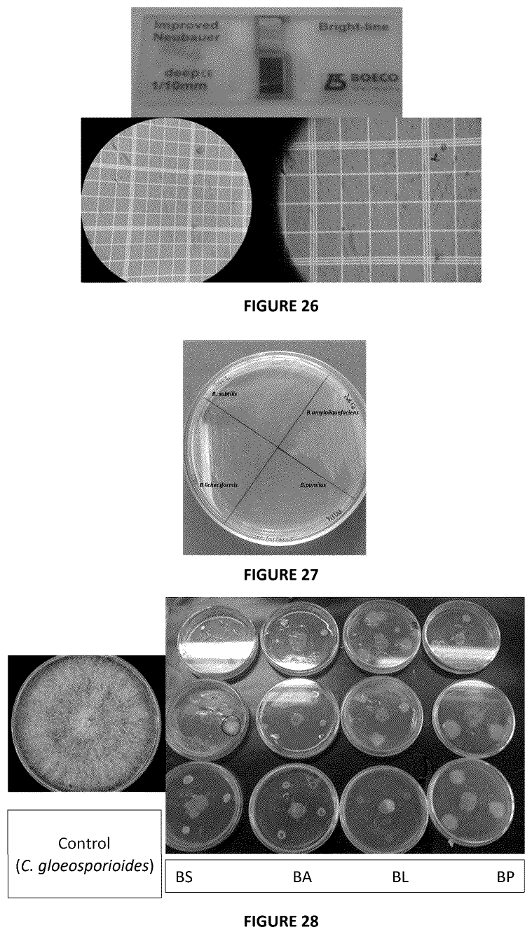

For the preparation of the beads or spheres, the cell suspension of the bacteria was mixed with the Alginate-Starch solution, which was later dripped onto a 1.5 M calcium chloride (CaCl₂)) solution using a glass burette. Previously sterilized with a capacity of 25 mL and performed the procedure constantly. The solution was left under constant stirring at 110 rpm for 30 minutes to achieve total cross-linking of the alginate by the action of CaCl₂), allowing the formation of beads/spheres containing the microbial consortium. Subsequently, five beads were added in sterile vials with 5 mL of 5% sodium citrate to determine the microbial concentration obtained after encapsulation. The vials were vortexed for 2 minutes to allow the dissolution of the matrix and to count viable microorganisms using the technique of a Neubauer chamber FIG. 26 (Ramirez et al., 2018).

The Neubauer Chamber is an instrument used in cell culture to count cells in a liquid culture medium, in this case, used to count bacterial cells in alginate beads/spheres. It consists of two glass plates, between which a known volume of liquid can be accommodated; in this case, it was 20 microliters.

TABLE 19

| Bead/Sphere | Final Concentracion (cells/mL) |
|---|---|
| 1 | 1.2 × 10⁵ |
| 2 | 1.0 × 10⁵ |
| 3 | 1.3 × 10⁵ |
| 4 | 1.0 × 10⁵ |
| 5 | 1.15 × 10⁵ |

As can be seen, the variability of the concentration in the alginate beads was considerable since they are not homogeneous in size and shape; however, survival was high, especially in bead number 5. This technique did not allow the inventors to differentiate which bacterium was in the highest concentration. A more significant amount of bacilli was observed.

### Conclusions

The solubilization of potassium and other minerals are other functions of Azotobacter in the soil that promote plant growth.

The mixture of Azotobacter with Rhizobium has also been positive. Azotobacter produces hormones increasing root growth and make more roots available for Rhizobium infection with more nodulation, nitrogen fixation, and higher yield.

Another bacterium associated with Azotobacter is Azospirillum. According to the bibliography, they have given excellent results when both bacteria have been inoculated together in strawberry, mustard, potato, paprika, and wheat cultures, In addition, these two bacteria together have been shown to improve the effect in plants against salinity stress.

The use of microbial alginate to encapsulate inoculants is attractive for new formulations. More tests are needed to evaluate the survival of the bacteria and its effectiveness in producing secondary metabolites.

### Example 8

A complex microbial consortium was established, made up of 3 bacteria of the Bacillus genus; 3 probiotic Gram-positive bacteria of the genus Lacticaseibacillus, including L. casei, L. paracasei, and L. rhamnosus, which are non-sporulating, non-motile bacilli having various applications (e.g., food fermentation); and one bacterium of the genus Burkolderia (B. tropica), being isolated from the rhizoplane of maize plants, and identified as a Gram-negative, mobile, aerobic, and considered a plant growth-promoting bacterium (BPCV). Leaf extracts from the tobacco plant or wild tobacco (Nicotiana glauca) were supplemented with Ethylhexyl glycerin as an adjuvant and used as a vehicle.

In a laboratory test, the inventors evaluated the in vitro confrontation between bacteria and pathogenic fungi Penicillium sp. and Aspergillus sp. Bacterial inoculums were prepared at the following concentrations:

TABLE 20
Composition 1 and concentrations of bacteria

| Strain | Concentration |
|---|---|
| Bacillus subtilis Q135 | 1 × 10⁸ |
| Bacillus amyloliquefaciens | 1 × 10⁶ |
| Bacillus licheniformis | 1 × 10⁵ |
| Burkolderia tropica | 1 × 10⁷ |
| Lacticaseibacillus casei | 2 × 10² |
| Lacticaseibacillus paracasei | 2 × 10² |
| Lacticaseibacillus rhamnos | 2 × 10³ |

The bacterial inoculum's highest in vitro antagonistic activity was with the Bacillus and Lacticaseibacillus strains, obtaining a decrease in the growth of Penicillium sp. by 75% and 63%, respectively, and in Aspergillus sp. with 68% and 72%. Concerning the non-control inoculated, this inhibition can be improved, increasing the concentration of the inoculum by one order, to further increase the production of hydrolytic enzymes. The Burkolderia tropica inoculum showed relatively low antagonistic activity. It decreased 16% of the growth of Penicillium sp. and 12% in Aspergillus sp. Despite the high cell concentration used with Burkolderia tropica, this bacterium seems to have lower amounts of metabolites or bioactive substances to control pathogenic fungi. The fungi equaled the growth of the control not being inoculated with bacterial inoculum, in less than 48 hours.

The wild tobacco extracts, used as vehicles in the pre-formula, were prepared by macerating approximately 300 gr of healthy and green leaves in 1 liter of water to obtain the 100% concentrated macerate. Subsequently, a dilution with sterile distilled water was carried out until reaching a 3% concentration, supplemented with 1.8% Ethylhexyl glycerin to increase the viscosity of the pre-formula.

Subsequently, the bacterial inoculums were added to the wild tobacco extract at the same concentrations evaluated in the in vitro assay to determine their antagonistic action on avocado fruits affected by the pathogen Alternaria sp. After approximately 72 hours, the pre-formula decreased pathogen growth by 11.3% only.

These results may be attributed to a wide variety of secondary metabolites synthesized by wild tobacco plants which may have been further activated by the maceration and incubation process. Affecting the viability of microbial consortia decreases the cell population and therefore, considerably affects the colonization and establishment of the inoculum on the surface of the fruits.

The concentration of Ethylhexyl glycerin was not ideal either because the viscosity was not sufficient for adequate adhesion on the fruit.

TABLE 21
Composition 2 and concentrations of bacteria

| | | Concentration | Concentration | |
|---|---|---|---|---|
| | | NOT efficient | Efficient | Contents |
| Formulation | bacterial inoculum | (CFU/mL⁻¹) | (CFU/mL⁻¹) | total % |
| Bacteria | Bacillus subtilis Q135 | 1 × 10⁸ | 1 × 10⁸ | 95.2 |
| | Bacillus amyloliquefaciens | 1 × 10⁶ | 1 × 10⁷ | |
| | Bacillus licheniformis | 1 × 10⁵ | 1 × 10⁶ | |
| | Burkolderia tropica | 1 × 10⁷ | None | |
| | Lacticaseibacillus casei | 2 × 10² | 2 × 10³ | |
| | Lacticaseibacillus | 2 × 10² | 2 × 10³ | |
| | paracasei | | | |
| | Lacticaseibacillus | 2 × 10³ | 2 × 10⁴ | |
| | rhamnosus | | | |
| Vehicle | Wild tobacco (Nicotiana | 3% | None | 4.8 |
| | glauca) | | | |
| | Ethylhexyl glycerin | 1.8% | 2.5% | |

Four more bacteria were added to the composition. One species of each genus of Latilactobacillus, Lactiplantibacillus, Lactococcus, and Limosilactobacillus, for a total of ten bacteria of different genera and species (Table 22). Microbial consortia allow complementary physiological processes that interact to form more efficient, stable, and rapidly colonizing microbial populations in fruits and vegetables. Extracts from custard apple leaves (Annona cherimola) were used as vehicles, supplemented with sodium hydroxide and Ethylhexyl glycerin.

In the antagonistic evaluation of the pre-formula, bacterial inoculums were used in the order of 1×10⁶-1×10⁸ CFU/mL for Bacillus species and 2×10⁵-2×10⁶ CFU/mL for Lactobacillus bacteria.

Chirimoya leaves were mixed with a 5% g/g sodium hydroxide solution in a mass ratio of 1:1.5, allowed to digest for one hour, and subsequently diluted in sterile distilled water until reaching a concentration of 28%. The 2.5% Ethylhexyl glycerin product was also added as an adjuvant.

Determining the effectiveness of the formula, different groups of Hass variety avocado fruits were treated. All avocado fruits were stored in cold rooms, one group with a commercial product (fungicide), another group with the 3% pre-formula, and a final control group (no treated). After seven weeks of storage, the efficacy of formula four was quantified, and it did not significantly reduce the amount of rotten fruit during storage. The commercial product reduced the loss of fruits due to rot by 82%, while the pre-formula only reduced by 15%. That is, 85% of the treated fruits were lost.

Subsequently, it was determined that the chirimoya extracts, together with sodium hydroxide, produced cell lysis in most bacteria, especially the Bacillus genus, because the matrix of the pre-formula was alkalinized. They reached a pH of 9.6, which considerably affects bacterial cells' osmotic pressure. On the other hand, the formation of Bacillus endospores was not enough to effectively colonize the fruit and maintain itself over time.

TABLE 22
Composition 3 (and concentrations of bacteria) and carrier 1

| | | Concentration | Concentration | |
|---|---|---|---|---|
| | | NOT efficient | Efficient | Contents |
| Formulation | bacterial inoculum | (CFU/mL⁻¹) | (CFU/mL⁻¹) | total % |
| Bacteria | Bacillus subtilis Q135 | 1 × 10⁸ | 1 × 10⁸ | 64.5 |
| | Bacillus amyloliquefaciens | 1 × 10⁶ | 1 × 10⁶ | |
| | Bacillus licheniformis | 1 × 10⁶ | 1 × 10⁵ | |
| | Lacticaseibacillus casei | 2 × 10⁵ | 2 × 10⁵ | |
| | Lacticaseibacillus | 2 × 10⁵ | 2 × 10⁴ | |
| | paracasei | | | |
| | Lacticaseibacillus | 2 × 10⁴ | 2 × 10⁴ | |
| | rhamnosus | | | |
| | Latilactobacillus sakei | 2 × 10⁴ | 2 × 10⁵ | |
| | Lactiplantibacillus | 2 × 10⁶ | 2 × 10⁶ | |
| | plantarum | | | |
| | Lactococcus lactis | 2 × 10⁵ | 2 × 10⁵ | |
| | Limosilactobacillus | 2 × 10⁶ | 2 × 10⁵ | |
| | fermentum | | | |
| Vehicle | Chirimola (Annona | 28% | None | 35.5 |
| | cherimola) | | | |
| | Sodium hydroxide | 5% | None | |
| | Ethylhexyl glycerin | 2.5% | 2.5% | |

Next, the inventors examined the above-disclosed microbial consortium (as in Table 21) with a different vehicle.

Specifically, the composition of the pre-formulation kept the same microbial consortium since it has been evaluated in the previous pre-formulas and different concentrations, showing specific stability and proven functionality in vitro tests. The maximum concentration used was 1× 10⁸ CFU/mL in Bacillus, and the lowest concentration was 2×10⁴ CFU/mL in Lacticaseibacillus rhamnosus.

The vehicle tested in this pre-formulation was composed of citrus fruit extracts. Citrus Aurantium Amara (bitter orange) at 2.2% and Citrus reticulata (tangerine) 1.5%, supplemented with Ethylhexyl glycerin at 2.5%; these botanical extracts have the potential to be exploited as innovative substrates to protect and increase the stability of bacterial inoculum in biological control products.

The formula (as in Table 23) was evaluated on Persimmon fruits stored in freezers for ten weeks to determine its effect on the extension of fruit shelf life. For this, a group of fruits (preserved) were sprinkled with the 3% pre-formula. Another group was left as a control (not inoculated). Later, they were stored in cold chambers to evaluate the appearance of black spots caused by Alternaria alternata.

The results showed that the pre-formula reduced approximately 42% of the spots, offering a lower severity concerning the control group. It indicated that improving some components of the pre-formula could obtain significant data on the extension of the useful life of the persimmon fruit. However, later it was determined that the concentration of some Lactobacillus bacteria was not the most appropriate since the microbial mass of some was below 1 mg/mL.

TABLE 23
Composition 3 (and concentrations of bacteria) and carrier 2

| | | Concentration | Concentration | |
|---|---|---|---|---|
| | | NOT efficient | Efficient | Contents |
| Formulation | bacterial inoculum | (CFU/mL⁻¹) | (CFU/mL⁻¹) | total % |
| Bacteria | Bacillus subtilis Q135 | 1 × 10⁸ | 1 × 10⁸ | 93.8 |
| | Bacillus amyloliquefaciens | 1 × 10⁶ | 1 × 10⁶ | |
| | Bacillus licheniformis | 1 × 10⁶ | 1 × 10⁵ | |
| | Lacticaseibacillus casei | 2 × 10⁵ | 2 × 10⁵ | |
| | Lacticaseibacillus | 2 × 10⁵ | 2 × 10⁴ | |
| | paracasei | | | |
| | Lacticaseibacillus | 2 × 10⁴ | 2 × 10⁴ | |
| | rhamnosus | | | |
| | Latilactobacillus sakei | 2 × 10⁴ | 2 × 10⁵ | |
| | Lactiplantibacillus | 2 × 10⁶ | 2 × 10⁶ | |
| | plantarum | | | |
| | Lactococcus lactis | 2 × 10⁵ | 2 × 10⁵ | |
| | Limosilactobacillus | 2 × 10⁶ | 2 × 10⁵ | |
| | fermentum | | | |
| Vehicle | Citrus Aurantium Amara | 2.2% | 2.56 | 6.2 |
| | Citrus reticulata | 1.5% | 2.56 | |
| | Ethylhexyl glycerin | 2.5% | 2.5% | |

The inventors have compared various aspects attributed to the bacterial consortia as disclosed herein, in effective and non-effective concentrations (Table 23). These attributes included: bacterial biomass (mg/ml), adhesion, metabolites (antibiosis), fermentation, polysaccharides, compatibility, etc.

TABLE 24

| | | | | Concentration | | |
|---|---|---|---|---|---|---|
| | | Concentration NOT | | efficient | | Content total |
| Formulation | Bacterial inoculum | efficient (CFU/mL) | Observation | (CFU/mL) | Observation | % |
| Composition 3 | Bacillus subtilis | 1 × 10⁸ | <1 mg/mL | 1 × 10¹⁰ | >2 mg/mL bacterial | 26 |
| | | | bacterial biomass | | biomass | |
| | Bacillus | 1 × 10⁵ | <50% adhesion | 1 × 10⁸ | >70% adhesion | 22 |
| | amyloliquefaciens | | | | | |
| | Bacillus | 1 × 10⁴ | <30% metabolites | 1 × 10⁶ | >50% metabolites | 18 |
| | licheniformis | | (antibiosis) | | (antibiosis) | |
| | Lacticaseibacillus | 2 × 10² | <0.8 mg/mL | 2 × 10⁴ | >1.5 mg/mL | 2.6 |
| | casei | | bacterial biomass | | bacterial biomass | |
| | Lacticaseibacillus | 2 × 10² | <1 mg/mL | 2 × 10⁴ | >2.2 mg/mL | 2.2 |
| | paracasei | | bacterial biomass | | bacterial biomass | |
| | Lacticaseibacillus | 2 × 10⁴ | <50% C | 2 × 10⁶ | >90% C | 2.2 |
| | rhamnosus | | fermentation | | fermentation | |
| | Latilactobacillus | 2 × 10⁴ | <45% | 2 × 10⁶ | >90% | 2.2 |
| | sakei | | polysaccharides | | polysaccharides | |
| | Lactiplantibacillus | 2 × 10⁵ | <1.3 mg/mL | 2 × 10⁷ | >2.5 mg/mL | 4.6 |
| | plantarum | | bacterial biomass | | microbial biomass | |
| | Lactococcus lactis | 2 × 10⁵ | <60% | 2 × 10⁷ | 100% compatibility | 4.6 |
| | | | compatibility | | | |
| | Limosilactobacillus | 2 × 10² | <1 mg/mL | 2 × 10⁵ | >2.4 mg/mL | 2.8 |
| | fermentum | | microbial biomass | | bacterial biomass | |
| Composition 4 | Bacillus subtilis | 1 × 10⁸ | <1 mg/mL | 1 × 10¹⁰ | >2 mg/mL | 26 |
| | | | bacterial biomass | | bacterial biomass | |
| | Bacillus | 1 × 10⁵ | <50% adhesion | 1 × 10⁸ | >70% adhesion | 20 |
| | amyloliquefaciens | | | | | |
| | Bacillus | 1 × 10⁴ | <30% | 1 × 10⁶ | >50 | 18 |
| | licheniformis | | polysaccharides | | % polysaccharides | |
| | Lacticaseibacillus | 2 × 10² | <0.8 mg/mL | 2 × 10⁴ | >1.5 mg/mL | 2.6 |
| | casei | | bacterial biomass | | bacterial biomass | |
| | Lacticaseibacillus | 2 × 10² | <1 mg/mL | 2 × 10⁴ | >2.2 mg/mL | 2.2 |
| | paracasei | | bacterial biomass | | bacterial biomass | |
| | Lacticaseibacillus | 2 × 10⁴ | <50% | 2 × 10⁶ | >90% | 2.2 |
| | rhamnosus | | compatibility | | compatibility | |
| | Latilactobacillus | 2 × 10⁴ | <45% C | 2 × 10⁶ | >90% C | 2.2 |
| | sakei | | fermentation | | fermentation | |
| | Lactiplantibacillus | 2 × 10⁵ | <1.3 mg/mL | 2 × 10⁷ | >2.5 mg/mL | 2.6 |
| | plantarum | | bacterial biomass | | microbial biomass | |
| | Lactococcus lactis | 2 × 10⁵ | <60% | 2 × 10⁷ | 100% compatibility | 2.2 |
| | | | compatibility | | | |
| | Limosilactobacillus | 2 × 10² | <1 mg/mL | 2 × 10⁵ | >2.4 mg/mL | 2.8 |
| | fermentum | | microbial biomass | | bacterial biomass | |
| | Rhizobium tropici | 1 × 10⁶ | <0.7 mg/mL | 1 × 10⁸ | >2.0 mg/mL | 2 |
| | [symbol] | | [symbol] | | [symbol] | |
| | Rhizobium etli | 1 × 10⁶ | <45% | 1 × 10⁸ | >50% metabolites | 2 |
| | AM015 (*) | | metabolites | | (antibiosis) | |
| | Azospirillumsp | 1 × 10⁴ | <50% enzymes | 1 × 10⁶ | >80% enzymes | 2.4 |

| | | Concentration | | Concentration | | Content |
|---|---|---|---|---|---|---|
| Formulation | Bacterial inoculum | NOT efficient | Observation | efficient | Observation | total % |
| Composition 5 | Bacillus subtilis | 1 × 10⁸ | <1 mg/mL bacterial biomass | 1 × 10¹⁰ | >2 mg/mL bacterial | 24 |
| | | | | | [symbol] | |
| | Bacillus amyloliquefaciens | 1 × 10⁵ | <50% adhesion | 1 × 10⁸ | >70% adhesion | 20 |
| | Bacillus licheniformis | 1 × 10⁴ | <30% polysaccharides | 1 × 10⁶ | >50 | 18 |
| | | | | | [symbol] | |
| | Lacticaseibacillus casei | 2 × 10² | <0.8 mg/mL bacterial biomass | 2 × 10⁴ | >1.5 mg/mL bacterial | 2.6 |
| | | | | | biomass | |
| | Lacticaseibacillus paracasei | 2 × 10² | | 2 × 10⁴ | | 2.2 |
| | Lacticaseibacillus | 2 × 10⁴ | <1 mg/mL bacterial biomass | 2 × 10⁶ | >2.2 mg/mL bacterial | 2.2 |
| | rhamnosus | | | | biomass | |
| | Latilactobacillus sakei | 2 × 10⁴ | <50% compatibility | 2 × 10⁶ | >90% compatibility | 2.2 |
| | Lactiplantibacillus | 2 × 10⁵ | <45% C fermentation | 2 × 10⁷ | >90% C | 2.6 |
| | [symbol] | | | | [symbol] | |
| | Lactococcus lactis | 2 × 10⁵ | <1.3 mg/mL bacterial biomass | 2 × 10⁷ | >2.5 mg/mL | 2.2 |
| | | | | | [symbol] | |
| | Limosilactobacillus | 2 × 10² | <60% compatibility | 2 × 10⁵ | 100% compatibility | 1.4 |
| | [symbol] | | | | | |
| | Rhizobium tropiciAM001 | 1 × 10⁶ | <1 mg/mL microbial biomass | 1 × 10⁸ | >2.4 mg/mL bacterial | 2 |
| | | | | | [symbol] | |
| | Rhizobium etliAM015 | 1 × 10⁶ | <0.7 mg/mL microbial | 1 × 10⁸ | >2.0 mg/mL bacterial | 2 |
| | | | biomass | | biomass | |
| | Azospirillumsp AS101 | 1 × 10⁴ | <45% metabolites | 1 × 10⁶ | >50% metabolites | 2.4 |
| | Azotobacter vinelandii | 1 × 10⁶ | <60% enzymes and | 1 × 10⁹ | >80% enzymes and | 3.4 |
| | AS122 (***) | | hormones; <56% metabolites | | hormones; >84% | |
| | | | (antibiosis); <20% alginate | | metabolites | |

[symbol]

indicates data missing or illegible when filed

The inventors have compared various aspects attributed to the bacterial consortia as disclosed herein, in effective and non-effective concentrations (Table 24), when formulated with different carriers, as disclosed herein. These attributes included: pH, cell lysis, carbohydrate intake, adherence, conservation of metabolites, etc.

TABLE 25

| | | Concentration | | | | |
|---|---|---|---|---|---|---|
| | | NOT | | Concentration | | |
| | | efficient | | efficient | | Total content |
| Carrier | Vehicles | (%) | Observation | (%) | Observation | (%) |
| 1 | Citrus | 4.56 | pH < 2.4 | 2.56 | pH > 3.3-3.8 | 2.56 |
| | Aurantium | | (very acid) | | (tolerant) | |
| | Amara (bitter | | | | | |
| | orange) | | | | | |
| | Citrus | 1.25 | >63% cell | 2.56 | <15% | 2.56 |
| | reticulata | | lysis | | cell lysis | |
| | (tangerine) | | | | | |
| | Citrus | 1 | <55% | 1.8 | >80% | 1.8 |
| | aurantium | | carbohydrate | | carbohydrate | |
| | sinensis | | intake | | intake | |
| | (sweet | | | | | |
| | orange) | | | | | |
| | Ethylhexyl | 2.5 | <46% | 3.4 | 98% | 3.4 |
| | glycerin | | adherence | | adherence | |
| | Phenoxyethanol | 1.5 | <52% | 2.48 | >90% | 2.48 |
| | | | conservation | | conservation | |
| | | | of metabolites | | of metabolites | |
| 2 | Citrus | 4.56 | pH < 2.4 | 2.56 | pH > 3.3 | 1.4 (*) |
| | Aurantium | | (very acid) | | 3.8 | |
| | Amara (bitter | | | | (tolerant) | |
| | orange) | | | | | |
| | Citrus | 1.25 | >43% cell | 2.56 | <8% cell | 1.5 |
| | reticulata | | lysis | | lysis | |
| | (tangerine) | | | | | |
| | Citrus | 1 | <55% | 1.8 | >80% | 1.8 |
| | aurantium | | carbohydrate | | carbohydrate | |
| | sinensis | | intake | | intake | |
| | (sweet | | | | | |
| | orange) | | | | | |
| | Ethylhexyl | 2.5 | <46% | 3.4 | 98% | 3.4 |
| | glycerin | | adherence | | adherence | |
| | Phenoxyethanol | 1 | <52 | 2.48 | 100% | 2 |
| | | | conservation | | conservation | |
| | | | of metabolites | | of metabolites | |
| | Brassica | 1.5 | pH | 2.7 | pH, | 2.7 |
| | oleraceae var. | | Unstable < | | stable > | |
| | capitata | | 75% | | 90% | |
| | (Glucosinolates) ** | | glucosinolates; <30% | | glucosinolates; >50% | |
| | | | myrosinase | | myrosinase | |
| | | | enzyme | | enzyme | |
| 3 | Citrus | 4.56 | pH < 2.4 | 2.56 | pH > 3.3-3.8 | 1.4 |
| | Aurantium | | (very acid) | | (tolerant) | |
| | Amara (bitter | | | | | |
| | orange) | | | | | |
| | Citrus | 1.25 | >43% cell | 2.56 | <8% cell | 1.5 |
| | reticulata | | lysis | | lysis | |
| | (tangerine) | | | | | |
| | Citrus | 1 | <55% | 1.8 | >80% | 1.8 |
| | aurantium | | carbohydrate | | carbohydrate | |
| | sinensis | | intake | | intake | |
| | (sweet | | | | | |
| | orange) | | | | | |
| | Ethylhexyl | 1 | <65% | 3.4 | 100% | 1.4 |
| | glycerin | | adherence | | adherence | |
| | Phenoxyethanol | 1 | <52% | 2.48 | 100% | 2 |
| | | | conservation | | conservation | |
| | | | of metabolites | | of metabolites | |
| | Brassica | 1.5 | pH | 2.7 | pH | 2.7 |
| | oleraceae var. | | Unstable, <75% | | stable, >90% | |
| | capitata | | glucosinolates | | glucosinolates | |
| | (Glucosinolates) | | | | | |
| | Microbial | 1.2 | <62% | 2 | >95% | 2 |
| | alginate (***) | | alginate, | | alginate, | |
| | | | larger | | minor | |
| | | | particles | | particles | |

The vehicles represent 12.8% of the total composition of each formulation.
(*) Synergism and complementarity between the vehicles allow the stability of the matrix, despite the changes in the amounts of %.
(**) Brassica oleraceae var. Capitata, the extracts together with the enzyme myrosinase, restrict the growth of fungi and exhibit immunosuppressive and anticancer properties.
(***) The only prokaryotic source of alginate, more stable, allows control of the gas exchange, moisture permeation, or oxidative processes.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A synthetic composition comprising:
a. bacterial consortium comprising:
i. at least three bacterial species belonging to the genus Bacillus; and
ii. at least three bacterial species belonging to the genus Lacticaseibacillus; and
b. an acceptable carrier.

2. The synthetic composition of claim 1, wherein said at least three bacterial species belonging to the Bacillus, comprise: Bacillus subtilis, Bacillus amyloliquefaciens, and Bacillus licheniformis.

3. The synthetic composition of claim 1, wherein said at least three bacterial species belonging to the genus Lacticaseibacillus, comprise: Lacticaseibacillus casei, Lacticaseibacillus paracasei, and Lacticaseibacillus rhamnosus.

4. The synthetic composition of claim 1, further comprising at least one first additional bacterial species belonging to any one of:
a. the genus Latilactobacillus;
b. the genus Lactiplantibacillus;
c. the genus Lactococcus;
d. the genus Limosilactobacillus; and
e. any combination of (a) to (d).

5. The synthetic composition of claim 4, wherein any one of:
a. said at least one first additional bacterial species belonging to the genus Latilactobacillus, comprises Latilactobacillus sakei;
b. said at least one first additional bacterial species belonging to the genus Lactiplantibacillus, comprises Lactiplantibacillus plantarum;
c. said at least one first additional bacterial species belonging to the genus Lactococcus, comprises Lactococcus lactis; and
d. said at least one first additional bacterial species belonging to the genus Limosilactobacillus, comprises Limosilactobacillus fermentum.

6. The synthetic composition of claim 1, further comprising at least one second additional bacterial species belonging to any one of:
a. the genus Rhizobium;
b. the genus Azospirillum;
c. the genus Azobacter; and
d. any combination of (a) to (c),
and optionally wherein any on of:
a. said at least one second additional bacterial species belonging to the genus Rhizobium, comprises Rhizobium tropici AM001, Rhizobium etli AM015, or both;
b. said at least one second additional bacterial species belonging to the genus Azospirillum, comprises Azospirillum sp AS101; and
c. said at least one second additional bacterial species belonging to the genus Azobacter, comprises Azobacter vinelandii AS122.

7. (canceled)

8. The synthetic composition of claim 1, comprising said at least three bacterial species belonging to the genus Bacillus and said at least three bacterial species belonging to the genus Lacticaseibacillus at colony forming unit (CFU) to CFU ratio ranging from 10,000:1 to 1,000:1.

9. The synthetic composition of claim 2, comprising said Bacillus subtilis, Bacillus amyloliquefaciens, and Bacillus licheniformis, at a CFU ranging from 1×10⁹ to 1×10¹², 1×10⁶ to 1×10¹⁰, and 1×10⁵ to 1×10⁸, respectively.

10. The synthetic composition of claim 3, comprising Lacticaseibacillus casei, Lacticaseibacillus paracasei, and Lacticaseibacillus rhamnosus, at a CFU ranging from 1×10³ to 1×10⁶, 1×10³ to 1×10⁶, and 1×10⁵ to 1×10⁸, respectively.

11. The synthetic composition of claim 5, comprising any one of:
a. Latilactobacillus sakei at a CFU ranging from 1×10⁵ to 1×10⁸,
b. Lactiplantibacillus plantarum at a CFU ranging from 1×10⁶ to 1×10⁹;
c. Lactococcus lactis at a CFU ranging from 1×10⁶ to 1×10⁹; and
d. Limosilactobacillus fermentum at a CFU ranging from 1×10⁴ to 1×10⁷.

12. The synthetic composition of claim 6, comprising any one of:
a. Rhizobium tropici AM001, Rhizobium etli AM015, or both at a CFU ranging from 1×10⁷ to 1×10¹⁰;
b. Azospirillum sp AS101 at a CFU ranging from 1×10⁵ to 1×10⁸; and
c. Azobacter vinelandii AS122 at a CFU ranging from 1×10⁷ to 1×10¹¹.

13. The synthetic composition of claim 6, comprising Rhizobium tropici AM001 and Rhizobium etli AM015 at a CFU: CFU ratio of 1:1, and optionally wherein said carrier comprises: Citrus Aurantium Amara extract, Citrus reticulata extract, Citrus aurantium sinensis extract, Ethylhexyl glycerin, Phenoxyethanol, or any combination thereof.

14. (canceled)

15. The synthetic composition of claim 13, wherein said carrier comprises: Citrus Aurantium Amara extract, Citrus reticulata extract, Citrus aurantium sinensis extract, Ethylhexyl glycerin, and Phenoxyethanol.

16. The synthetic composition of claim 13, wherein said carrier further comprises any one of: Brassica oleraceae var. capitata extract, Microbial alginate, and both.

17. The synthetic composition of claim 13, further comprising a glycoside hydrolase, and optionally wherein said glycoside hydrolase is myrosinase.

18. (canceled)

19. The synthetic composition of claim 13, comprising said carrier in a weight per weight ratio (w/w) ranging from 8% to 15% of said composition.

20. The synthetic composition of claim 13, being: (i) a fungicidal composition; (ii) formulated for dipping, soaking, spraying, coating, or any combination thereof, to a plant or a plant part; or (iii) both (i) and (ii).

21. (canceled)

22. A method for preventing or treating a fungal infection in a plant or a plant part, the method comprising contacting said plant or plant part with an effective amount of the synthetic composition of claim 13, thereby preventing or treating a fungal infection in the plant or plant part.

23. The method of claim 22, wherein any one of: (i) said fungal infection is induced by a fungus being selected form the group consisting of: Botrytis cinerea, Mycosphaerella fragarie, Colletotrichum sp., Alternaria alternata, Penicillium italicum, Phytophthora citroththora, Penicillium digitatum, Phomopsis sp., Diplodia sp., Rhizopus nigricans, Sphaceloma persea, Cercospora purpura, Cladosporium herbarum, Neofabraea alba, Diplodia seriata, Phacidiopycnis washingtonensis, Rhizopus stolonifer/Mucor spp., Monilinia fructicola, Venturia inaequalis, Stemphillium sp., and any combination thereof; (ii) said plant part is selected from the group consisting of: a fruit, a leaf, a flower, a seed, a tuber, a bulb, and any combination thereof; (iii) said contacting comprises preharvest contacting, postharvest contacting, or both; (iv) said contacting is postharvest contacting, and optionally wherein said post harvesting contacting is in a storage facility; (v) said contacting is by dipping, soaking, spraying, coating, washing, humidifying, fogging, or any combination thereof, said plant or plant part with said composition; (vi) said preventing or treating comprises inhibiting any one of: attachment, germination, penetration, host tissue colonization, growth, proliferation, metabolism, spore release, hyphae production, hyphae penetration, or any combination thereof, of a fungus inducing said fungal infection; (vii) said preventing or treating comprises increasing shelf life of said plant or plant part by at least 20%; and (viii) any combination of (i) to (vii).

24. (canceled)

25. (canceled)

26. (canceled)

27. (canceled)

28. (canceled)

29. (canceled)

30. (canceled)


SEQUENCE LISTING (6 sequences)

SEQ ID NO: 1 - DNA, 20 bp, synthetic construct
  source 1..20: mol_type = other DNA
agagtttgatcctggctcag

SEQ ID NO: 2 - DNA, 16 bp, synthetic construct
  source 1..16: mol_type = other DNA
taccttgttacgactt

SEQ ID NO: 3 - DNA, 21 bp, synthetic construct
  source 1..21: mol_type = other DNA
gagagtttgatcctggctcag

SEQ ID NO: 4 - DNA, 20 bp, synthetic construct
  source 1..20: mol_type = other DNA
ctacggctaccttgttacga

SEQ ID NO: 5 - DNA, 17 bp, synthetic construct
  source 1..17: mol_type = other DNA
aaggaggtgatccagcc

SEQ ID NO: 6 - DNA, 19 bp, synthetic construct
  source 1..19: mol_type = other DNA
gagtttgatcctggctcag